(19) United States
(12) Patent Application Publication
(10) Pub. No.: US 2025/0000407 A1
(43) Pub. Date: Jan. 2, 2025

(54) PERSONALIZED BRAIN STATE GUIDANCE SYSTEM AND METHOD

(71) Applicant: Mark McDonough, Manchester, MA (US)

(72) Inventor: Mark McDonough, Manchester, MA (US)

(21) Appl. No.: 18/341,944

(22) Filed: Jun. 27, 2023

(51) Int. Cl. A61B 5/16; A61B 5/375; A61B 5/38; A61B 5/384; G16H 20/70; G16H 50/20

(52) U.S. Cl. CPC A61B 5/16; A61B 5/375; A61B 5/38; A61B 5/384; G16H 20/70; G16H 50/20; G16H 40/63

(57) ABSTRACT

Methods and systems for intelligent, personalized guidance and recommendations for achieving a desired brain state, such as during meditation. In one embodiment, brainwave activity for an individual can be used to predict which protocol has the greatest likelihood of serving as the most effective practice for that user. In another example, personalized brain state feedback can be generated and presented to the user that isolates different features or metrics and describes the user's performance with respect to each component. In another embodiment, users can review their brain activity scores and opt to create new protocols that target a smaller subset of the technique in order to tailor the development of their practice journey to their desired brain states.

### TECHNICAL FIELD

The present disclosure generally relates to methods and systems for developing predicting which brain state training protocol is most suited to an individual, and in particular to methods and systems for providing users with tailored recommendations and guidance to facilitate desired changes to their brain's electrical activity.

### BACKGROUND

In recent years, there has been significant uptake of meditation and related relaxation techniques as a means of alleviating stress and maintaining good health. Meditation offers a safe, effective, and relatively inexpensive intervention for reducing chronic stress and other stress-related disorders, in addition to improving cognition, mood, sleep, and general well-being. Despite its popularity, little is known about the neural mechanisms by which meditation works, and there remains a need for more rigorous investigations of the underlying neurobiology of individual brain states. Considering the wide range of possible meditation techniques and associated brain states, it may be appreciated that different practices will produce different electrophysiological effects—and that individuals with different neuropsychological profiles will respond differently to each practice style. Indeed, in some respects, individual responses to each practice can affect the suitability of a particular meditation technique for a given individual. A one-size-fits-all approach may not accommodate each person's unique cognitive demands and levels of experience. With the recognition that not every practice style is suitable for everyone, it becomes clear that a more personalized, intelligent approach to promoting brain health is needed.

There is a need in the art for a system and method that addresses the shortcomings discussed above.

### SUMMARY

In one aspect, a method for automatically identifying and generating personalized brain state protocol recommendations is disclosed. The method can include a first step of receiving, at a protocol recommendation system for a meditation application, a dataset representing brainwave activity for a first user in a meditative condition, and a second step of automatically accessing, at the protocol recommendation system, a plurality of meditation protocols including at least a first meditation protocol and a second meditation protocol, where each meditation protocol promotes a different type of meditation experience. In addition, the method can include a third step of calculating, by the protocol recommendation system, at least a first depth score characterizing a performance of the dataset with respect to the first meditation protocol and a second depth score characterizing a performance of the dataset with respect to the second meditation protocol, where the first depth score is higher than the second depth score. A fourth step includes presenting, via a user interface for the meditation application and in response to the first depth score being higher than the second depth score, a recommendation to the first user to implement the first meditation protocol.

In another aspect, a method for providing personalized brain state guidance is disclosed. The method can include a first step of receiving, at a brainwave guidance system for a meditation application, a first dataset representing brainwave activity for a first user during a first guided meditation session implemented based on a first meditation protocol, the first meditation protocol including at least a first rule and a second rule, and a second step of calculating, by the brainwave guidance system, a first score characterizing a performance of the first dataset with respect to the first rule and a second score characterizing a performance of the first dataset with respect to the second rule. In addition, the method can include a third step of presenting, via the meditation application, both the first score and the second score, and a fourth step of receiving, via a user interface for the meditation application, a first request to improve the first score. The method can further include a fifth step of automatically creating, at the meditation application and in response to the first request, a second meditation protocol including only the first rule, and a sixth step of initiating, at the meditation application, a second guided meditation session for the first user that is implemented based on the second meditation protocol.

In another aspect, a method for providing personalized meditation feedback is disclosed. The method can include a first step of receiving, at a brainwave feedback system for a meditation application, a first dataset representing brainwave activity for a first user during a first guided meditation session implemented based on a first meditation protocol, the first meditation protocol including at least a first rule and a second rule. A second step includes calculating, by the brainwave feedback system, a first score characterizing a performance of the first meditation dataset with respect to the first rule and a second score characterizing a performance of the first meditation dataset with respect to the second rule. In addition, the method can include a third step of presenting, via the meditation application, a dashboard including: a description of the first rule accompanied by the first score, and a description of the second rule accompanied by the second score.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIGS. 1A and 1B depict an overview of a process of generating personalized brain state protocol recommendations, according to an embodiment;

FIGS. 2A and 2B is a schematic diagram of an environment for implementing a brain state protocol recommendation system, according to an embodiment;

FIG. 3 depicts an example depth score comparison graph that can inform a user's selection of a protocol, according to an embodiment;

FIG. 4 is a flow chart of a process of generating personalized brain state protocol recommendations, according to an embodiment;

FIG. 5 depicts an example of a user interface in which brain activity is analyzed and feedback provided, according to an embodiment;

FIG. 6 is an example look-up table for interpretation of performance assessments for each rule in a protocol, according to an embodiment;

FIG. 7 is an example of a detailed brain activity feedback report generated by the proposed systems, according to an embodiment;

FIG. 8 is a flow chart of a process of evaluating and scoring brain activity, according to an embodiment;

FIGS. 9, 10, 11, and 12 are a sequence of illustrations of user interfaces depicting a process of providing personalized brain state guidance based on subsets of a protocol, according to an embodiment; and

FIG. 13 is a flow chart of a process of providing personalized brain state guidance, according to an embodiment.

### DESCRIPTION OF EMBODIMENTS

Systems and methods to facilitate and improve EEG-based brain state training are disclosed. In some examples, the embodiments enable a meditation training platform with outcomes far more effective than conventional programs by providing users with mechanisms by which to customize their experience, goals, rewards, and target brain states based on their desired style of practice, level of expertise, and type of brain. The proposed systems allow for significantly more accurate feedback as well as an expedited learning cycle that supports, guides, and intelligently reinforces the brain activity selected by a user. In one example, the system provides a pre-established group of twelve or more carefully crafted, unique protocols, with each protocol including an additional set of sub-protocols that target specific meditation experience levels. In one embodiment, the system provides over 20 protocols, including both traditional meditation-driven protocols and modern results-driven protocols. In yet other embodiments, and as will be described in greater detail further below, the system is configured to create individualized protocols for individuals or groups of individuals, enabling development of an endless variety of custom protocols and implementation options.

As a general matter, EEG or electroencephalography is a method by which spontaneous electrical activity of the brain. Thus, a “brainwave” captured via EEG techniques can refer to a kind of traceable neurophysiological energy in a living brain. These bio-signals detected by EEG have been shown to represent the postsynaptic potentials of pyramidal neurons in the neocortex and allocortex. Voltage fluctuations measured by an EEG bio-amplifier and electrodes allow for monitoring of a person's brain activity. As the electrical activity monitored by EEG originates in neurons in the underlying brain tissue, the recordings made by the electrodes on the surface of the scalp vary in accordance with their orientation and distance to the source of the activity (i.e., brain location). For example, a healthy human EEG will show certain patterns of activity which correlate with how awake a person is. The amplitude of brainwaves is normally measured using microvolt unit (μV). Traditionally, a low amplitudes fall under 20 μV, medium amplitudes between 20-50 μV, and high amplitude are greater than 50 μV. The standard range of frequencies one observes in humans are between 1 to 30 Hz and above, with amplitudes varying between 20 to 100 μV. These observed frequencies have been subdivided in various groups, including: alpha (8-8 Hz), beta (8-30 Hz), delta (0.5-4 Hz), theta (4-7 Hz), and gamma (+30 Hz). Furthermore, the recorded shapes of sinusoidal brainwaves have two main features: up and down, or down and up (i.e., in oscillation). Although the description below will primarily focus on the use of brainwaves captured via EEG, it should be understood that in different embodiments, the proposed embodiments can be implemented based on data obtained via other neuroimaging techniques, including but not limited to ECog, MEG, fMRI, etc.

Furthermore, electrodes—sensor elements that detect the EEG signals and converts them into electrical signals—can be positioned to acquire data from specific brain anatomy locations or areas. These areas can be identified by their relative positions: front, back, left, and right, as well as the cingulate cortex situated in the medial aspect of the cerebral cortex. With these directional identifiers, quadrants (front-left, front-right, back-left, back-right) for the brain areas can be used. An EEG then reflects correlated synaptic activity caused by post-synaptic potentials of cortical neurons. The electric potentials generated by single neurons are far too small to be picked up by an EEG; instead, EEG activity reflects the summation of the synchronous activity of thousands or millions of neurons that have similar spatial orientation, radial to the scalp. As noted above, scalp EEG activity shows oscillations at a variety of frequencies. Several of these oscillations have characteristic frequency ranges and spatial distributions and are associated with different states of brain functioning (e.g., waking and the various sleep stages). These oscillations represent synchronized activity over a network of neurons. The neuronal networks underlying some of these oscillations are understood, while many others are not.

For purposes of reference, the various embodiments described herein can include and implement some or all of the features, techniques, methods, systems, and any other aspects described in U.S. patent application Ser. No. 18/305,775 to McDonough, filed on Apr. 24, 2023 titled “Brain State Protocol Development and Scoring System and Method” (Attorney Ref No: 210-1003), and U.S. patent application Ser. No. 18/305,796 to McDonough, filed on Apr. 24, 2023 titled “Brain State Rule Generation and Scoring System and Method” (Attorney Ref No: 210-1004), both of which are collectively referred to as the McDonough applications throughout this disclosure, and both applications of which are incorporated by reference herein.

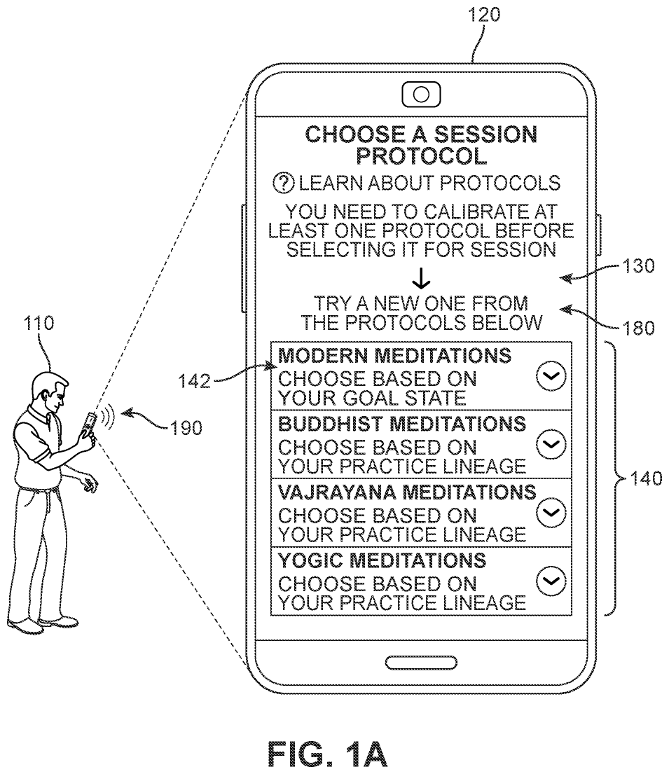

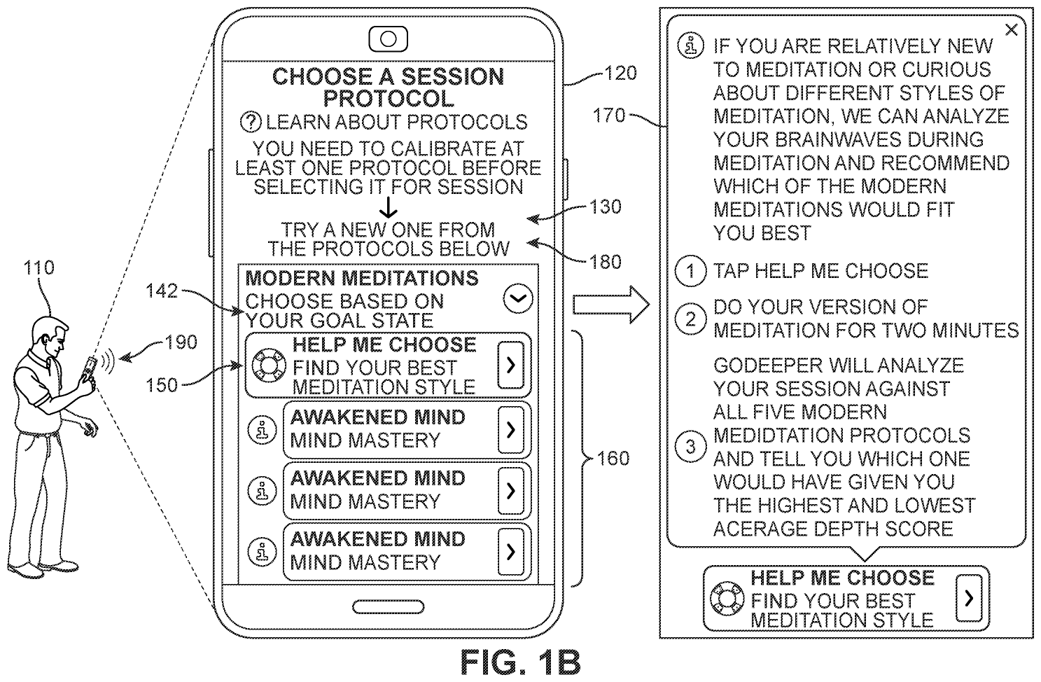

For purposes of introduction, an overview of one embodiment of the proposed systems and methods is illustrated with reference to FIGS. 1A and 1B. In FIG. 1A, an example of a brainwave protocol recommendation process is shown in which a first user 110 is accessing a meditation application (“application”) 180 via their client computing device (“client device”) 120 on which the application 180 can be installed or accessed over a network. In different embodiments, the client device 120 can include an electronics unit comprising a plurality of different components, such as a user interface component (e.g., a touchscreen display, keyboard, mouse, microphone, speaker, etc.), a user interface module, a processor, and/or a communication module. The client device 120 may include a system including one or more processors and memory. Memory may comprise a non-transitory computer readable medium. Instructions stored within memory may be executed by the one or more processors. The client device 120 may be configured to receive and analyze data from various input sensors associated the client device 120 or data that is communicated from external components or devices to client device 120.

Furthermore, a communication module may allow the client device 120 to communicate wirelessly. In this case, the communication module is illustrated as a wireless connection; however, wired connections may also be used. For example, the communication module may include a wired serial bus such as a universal serial bus or a parallel bus, among other connections. The communication module may also include a wireless connection using Bluetooth® radio technology, communication protocols described in IEEE 802.11 (including any IEEE 802.11 revisions), Cellular technology (such as GSM, CDMA, UMTS, EV-DO, WiMAX, or LTE), or Zigbee® technology, among other possibilities.

In different embodiments, the client device 120 includes a device display (“display”) that can, for example, present information and media for application 180. In one embodiment, client device 120 could operate in a client-server relationship with one or more servers of a remote/cloud-based computer system. In some cases, client device 120 may run client software through a web browser, in which case the client software may be hosted on a server associated with the computer system. In other cases, client device 120 may run client software in the form of a native software application that has been downloaded through a centralized marketplace (i.e., an “app store”). In some cases, while the client software that allows users to perform various tasks may be run on client device 120, the data may be retrieved from and stored on databases associated with the remote computer system.

In some embodiments, the first user 110 can receive and send information through a protocol selection user interface 130 for the monitoring app 180 that may be presented on the device display. In some embodiments, display may be a touchscreen, allowing the customer to interact with the user interface directly by touch. The user interface may refer to an operating system user interface or the interface of one or more software applications that may run on the client device 110. In some embodiments, the user interface can include a messaging window or other chat-space by which the user may review messages or other digital content.

In the example of FIG. 1A, the first user 110 can optionally be initially shown selectable meditation style category options 140 that can be used to narrow their selection to a particular group of meditation protocols, such as but not limited to Modern Meditations (e.g., shown here as a first option 142), as well as Buddhist Meditations, Vajrayana Meditations, and Yogic Meditations. These meditation style group names are aligned with the general practice traditions associated with the protocols in each group; however, it should be understood that these names can differ from what is shown in FIG. 1A.

In different embodiments, the monitoring app 180 can offer a user interface that may be accessed via any user computing device configured for connection to a network. In different embodiments, the application can be configured to offer content via native controls presented via an interface. Throughout this application, an “interface” may be understood to refer to a mechanism for communicating content through a client application to an application user. In some examples, interfaces may include pop-up windows that may be presented to a user via native application user interfaces (UIs), controls, actuatable interfaces, interactive buttons or other objects that may be shown to a user through native application UIs, as well as mechanisms that are native to a particular application for presenting associated content with those native controls. In addition, the terms “actuation” or “actuation event” refers to an event (or specific sequence of events) associated with a particular input or use of an application via an interface, which can trigger a change in the display of the application. This can include selections or other user interactions with the application, such as a selection of an option offered via a native control, or a ‘click’, toggle, voice command, or other input actions (such as a mouse left-button or right-button click, a touchscreen tap, a selection of data, or other input types).

Furthermore, a “native control” refers to a mechanism for communicating content through a client application to an application user. For example, native controls may include actuatable or selectable options or “buttons” that may be presented to a user via native application UIs, touch-screen access points, menus items, or other objects that may be shown to a user through native application UIs, segments of a larger interface, as well as mechanisms that are native to a particular application for presenting associated content with those native controls. The term “asset” refers to content that may be presented in association with a native control in a native application. As some non-limiting examples, an asset may include text in an actuatable pop-up window, audio associated with the interactive click of a button or other native application object, video or other media associated with a user interface, or other such information presentation. In some embodiments, a user can receive and send information through a user interface that may be presented on the device display. The user interface and display may represent a common component or may represent separate physical or functional components. In some embodiments, the display may be a touchscreen, allowing the customer to interact with the user interface directly by touch. The user interface may refer to an operating system user interface or the interface of one or more software applications that may run on the client device, such as the app.

As described in greater detail in the McDonough applications, in different embodiments, the proposed systems include protocols created to foster and promote specially directed brainwave training of individuals toward one or more pre-designated meditation techniques or approaches. These protocol groups can include Modern Meditations that encompass a group of meditation protocols such as: (1) Awakened Mind, (2) Calm & Relaxed (3) Creativity, (4) Focus, (5) Mindfulness/Stress Reduction, (6) Open Heart, (7) Quiet Mind. The predesignated style categories can further include Tradition-Based Meditations that include but are not limited to Buddhist Meditations including meditation protocols such as (8) Anapana (e.g., mindfulness of breathing), (9) Jhanas (e.g., single-pointed concentration leading to deep states of meditative absorption), (10) Kasina Meditation (e.g., a Samatha-like practice with a focus on kasinas (usually specific shapes, colors, or objects)), (11) Metta (e.g., radiating love and goodwill to ever wider circles of beings), (12) Samatha (e.g., breath/object focus practice for calmness and tranquility), (8) Traditional Mindfulness (e.g., noticing sensations & non-reactivity), (14) Vipassana/Goenka Body-Scanning (e.g., observing sensations in the body to develop tranquility and insight), (15) Vipassana/Thought-Watching (e.g., mindfully observing thoughts without being caught up in them), (16) Zazen/Focused Awareness (e.g., Third Ventricle), (17) Zazen/Open Monitoring (e.g., Shikantaza or “just sitting” to realize your true nature) as well as Vajrayana Meditations including protocols such as (18) Rigpa (e.g., nondual awareness beyond concepts), (19) Shamatha (e.g., breath/object focus practice for calmness and tranquility), (20) Yidam (e.g., Tantric practice involving visualization of an enlightened being), and Yogic Meditations including protocols such as (21) Acem Meditation (e.g., transcendental meditation derivative for relaxation and stress management), (22) Brahma Kumaris (BK) Raja Yoga (e.g., thought and visualization-based practices to inhabit soul consciousness), (23) Himalayan Yoga Tradition (e.g., mentally repeating a mantra with breath awareness), (24) Isha Yoga Shoonya (e.g., conscious non-doing to remove emotional and spiritual blocks, (25) Preksha Meditation (e.g., Jain meditation focusing on emotional purification and self-awareness), (26) Satyananda Yoga/Kaya Sthairyam (e.g., body steadiness), and (27) Satyananda Yoga/Japa (e.g., mantra recitation), and also Transcendental Meditations (TM) for transcending mantra recitation to experience blissful unbounded awareness such as (28) TM/Sidhi Practices to develop advanced TM practice (e.g., Samyama). Thus, for each of these broader meditation style groups, a different set of protocols—each protocol being associated with different rules and assigned weights—can be offered.

One example of such offerings is depicted in FIG. 1B, where in response to the first user's selection of the first option 142 for Modern Meditations, additional options 160 are presented by the application 180, including some of those mentioned above (e.g., Awakened Mind, Calm and Relaxed, Focus, etc.). It can be appreciated that in many cases, an individual meditation practitioner, whether new to the activity or highly experienced, can be uncertain regarding which meditation approach might represent the ‘best-fit’ or best align with their own brain state profile. In different embodiments, the proposed system can evaluate a person's unique brainwaves and determine which mediation technique is most likely to represent the best match for that person.

In other words, just as each person's neuropsychological profile can be complex and individualized, each person may be more receptive to one meditation technique over another. In cases where a person has been discouraged trying to practice one or more meditation approaches, finding they seem to be prone to distraction or cannot progress at the rate they were hoping they may desire to attain a target brain state that they are more likely to successfully to develop their expertise, the system can automatically recommend the program that is most appropriate based on their individual brain activity and pattern. Thus, with this context, it can be seen that one of the options shown in FIG. 1B is directed to a personalized recommendation tool 150 (“help me choose”). Once the user selects this option, additional information may be provided, as illustrated in expanded view 170, which encourages the user to implement the recommendation tool to help find the protocol for which the user has the greatest receptivity.

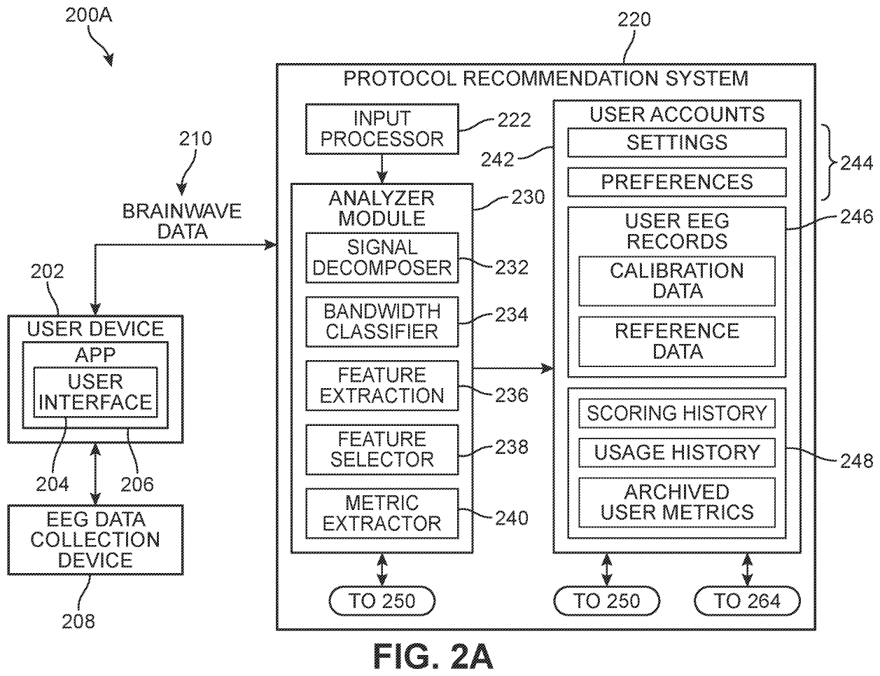

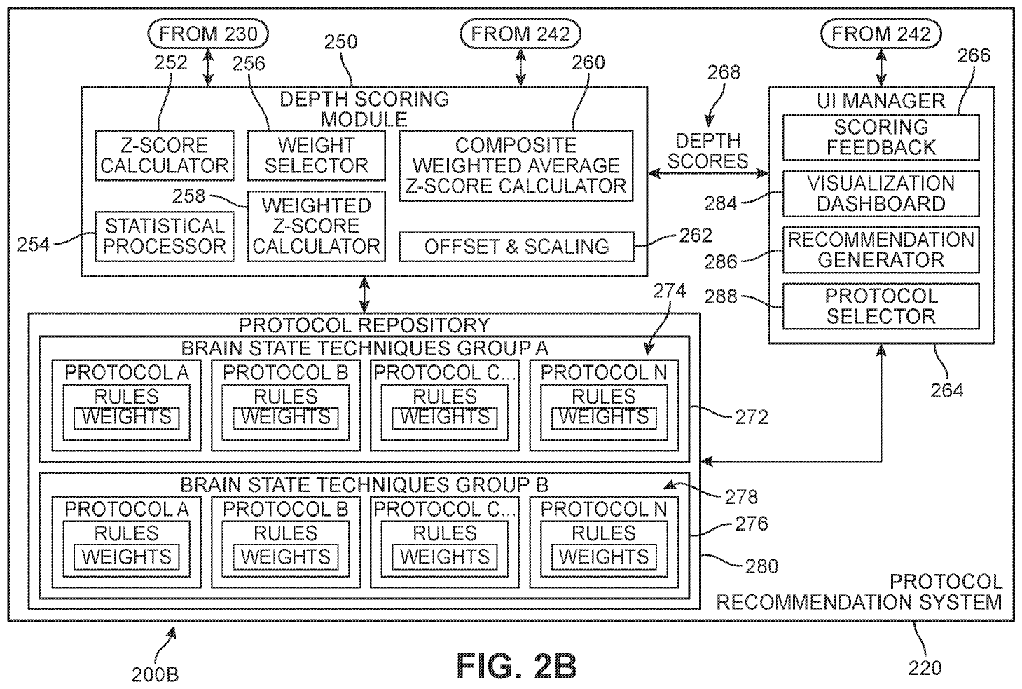

In order to provide the reader with a greater appreciation of the embodiments, FIGS. 2A and 2B depicts an overview of an embodiment of an environment 200 for implementation of a protocol recommendation system (“system”) 220 configured to provide a tailored scoring experience for each of the different styles of meditations at each of the levels of expertise, as described herein.

In contrast, the proposed embodiments are configured to receive EEG data and, across a wide spectrum of brain state goals and experience, identify the appropriate depth score. For example, in different embodiments, a user interface 206 for a meditation application (“app”) 204 running on a user computing device (“user device”) 202 can be in communication with an EEG or other brain-activity collection device 208 via a wired or wireless connection. In different embodiments, the user device 202 and other collection device 208 can also be configured to communicate with the system 220 over one or more network connections. Thus, in some embodiments, the various components of environment 200 can be accessed through a cloud network and/or stored on a cloud-based server, while in other embodiments some or all components described herein (including some or all modules of system 220) can reside locally in the user device and/or a remote server.

In different embodiments, networks could include one or more Wide Area Networks (WANs), Wi-Fi networks, Bluetooth or other Personal Area Networks, cellular networks, as well as other kinds of networks. It may be appreciated that different devices could communicate using different networks and/or communication protocols. The devices can include computing or smart devices as well as more simple IoT devices configured with a communications module/interface and a sensor. The communication module may include a wireless connection using Bluetooth® radio technology, communication protocols described in IEEE 202.11 (including any IEEE 202.11 revisions), Cellular technology (such as GSM, CDMA, UMTS, EV-DO, WiMAX, or LTE), or Zigbee® technology, among other possibilities. In many cases, the communication module is a wireless connection; however, wired connections may also be used. For example, the communication module may include a wired serial bus such as a universal serial bus or a parallel bus, among other connections. In addition, each client device can include provisions for communicating with, and processing information from, system 220. Each device may include one or more processors and memory. Memory may comprise a non-transitory computer readable medium. Instructions stored within memory may be executed by the one or more processors.

Thus, in different embodiments, brainwave data 210 such as EEG data obtained via collection device 208 from the user during a generic brain activity collection session (e.g., a person's meditation session or attempt thereof) can be received by system 220, whether the system 220 resides locally whether locally at the user device 202 or remotely over a network connection. This brainwave data 210 can represent the user's brain activity while engaging in their preferred or natural meditative condition. Thus, brainwave data 210 itself is not based on a specific guidance but is a reflection's the of the user's native, inherent, instinctive, or preferred meditative approach that can be recognized as their personal base state for meditation. In one example, an input processor 222 can receive the brainwave data 210 and prepare the data for use by the system 220. It should be appreciated that the brainwave data 210 can represent raw brain data such as EEG data, which is a complex waveform that includes brainwaves as well as artifacts such as electrical activity of nearby muscles, electrode motion interference, and/or ambient noise. Thus, in some embodiments, input processor 222 can be configured to ensure the data is filtered and pre-processed, and/or made ready for further analysis by downsampling, bandpass filtering, epoch of the data and removal of noisy epochs, removal of noisy components, general artifact rejection/suppression, etc. For example, in one embodiment, a proposed pre-processing pipeline can include several general stages, including filtering, an adaptive technique for artifact removal, interpolation, and independent component analysis (ICA) to remove the artifactual components.

The pre-processed brainwave data 210 is then received by an analyzer module 230. In some embodiments, a signal decomposer module 232 can perform extraction and separation of signal components from the composite signals and further clean the data for use by the other modules of the system 220. In addition, the clean brainwave data 210 can be initially segregated or otherwise classified for use by a bandwidth classification model 234 to determine specific types of information such as but not limited to bandwidth/frequency domain. In some embodiments, a feature extraction operation can initially be performed by a feature extraction module (“feature extractor”) 236.

For example, feature extraction can be performed using power spectral density (PSD) and/or log energy entropy. As a general matter, PSD represents the power distribution into the frequency component of the signal, and the latter describes the amount of information carried by a signal or how much randomness is in the signal. In one example, EEG signals can be transformed into PSD using the Fast Fourier Transform and one-second (or other time increment) hamming windows with sufficient overlap to maintain both temporal and frequency resolution and also to minimize the data loss in the window boundary. In addition, in some embodiments, the bandwidth classifier 234 can then divide each EEG channel into a plurality of sub-bands based on its frequency range (e.g., Delta, Theta, Alpha, Gamma, and Beta). As noted earlier, each EEG sub-band has a different frequency range, such that the average power spectrum for each sub-band can be calculated and used for further analysis. In some embodiments, for each sub-band, the average power spectral density ratios may also be calculated. As a non-limiting example, the average PSD of the beta band of each electrode in the frontal area can be divided by the alpha band of each electrode into parietal and occipital regions (frontal beta/parietal occipital alpha), beta divided by theta for each EEG electrode (beta/theta), and theta then divided by (alpha+beta) for each EEG electrode theta/(alpha+beta), and so forth for each of the other PSDs across the different sub-bands and locations.

It can be appreciated that feature extraction from PSD and/or log energy entropy of the EEG sub-bands can generate a large number of extracted features. In such instances, data complexity including the data variance can negatively impact the performance and accuracy of the system 220. Thus, in some optional embodiments, a feature selection module (“feature selector”) 238 can be included to reduce dimensionality, improve the predictive accuracy, and enhance the comprehensibility and usability of the obtained results. As some non-limiting examples, feature selector 230 can employ one or more feature selection algorithms, such as recursive feature elimination (RFE) and Lasso cross-validation (LassoCV) among others, to choose the most pertinent subset of the original features by automated removal of the irrelevant or redundant features.

In different embodiments, a metric extraction module (“metric extractor”) 240 can calculate and produce a plurality of metrics that can be used by a depth scoring module 250 to evaluate the user's recorded brain activity in the context of one or more previously created protocols. The process of protocol and rule generation was described in detail in the McDonough applications. In general, each protocol is defined by modulation of target values for specific metrics that have been identified as significant for a given meditation technique. These individual metric target values are each used to automatically create one rule for the given meditation technique. In some embodiments, a protocol can initially include 2-6 rules, though in other cases, only one rule may be created for implementation by a single protocol. Once the rules are compiled, the protocol can be offered for promoting the desired brain state training with a user. Thus, for purposes of this application, a protocol refers to an algorithm that defines—using one or more rules—a particular brain state based on values of metrics associated with that brain state. Each rule is used to guide a person toward the selected brain state, for example by reference to the absolute power of each frequency band (Alpha, Beta, etc.), the relative power of each frequency band, brainwave coherence, and brainwave complexity, as well as other metrics or features that have been detected when a person experiences that specific brain state.

Returning to FIG. 2A, in different embodiments, the metric extractor 240 enables the system 220 to organize the analyzed data and store values for brain-related metrics including, for each sub-band, power, percent of total power, power ratio, coherence, connectivity, minimum frequency, maximum frequency, phase synchrony, complexity, brain location, and target brainwave direction. In some embodiments, the analyzer module 230 generates values for data points across pre-specified equal time increments. Thus, the EEG data can be pre-segmented into multiple, equal time increments by the analyzer module 230 and, for each time increment of the multiple increments, identify the values of a plurality of metrics. In one example, the metrics are captured for each second, using a second-by-second segmentation of the data, though in other embodiments, the time increment selected can vary from less than a second to 10 or more seconds.

In different embodiments, the system 220 includes or is configured to access one or more databases, such as a user accounts database 242. The user accounts database 242 can include a content library that stores account data related to one or more users. The data may include, for each user, a username, a user profile, user selected settings and preferences 244 such as feedback thresholds, feedback type designations, brain training audio, language, subscription level, etc. Furthermore, each user account may further include a user EEG record repository 246 that stores previous EEG data, raw and/or processed and associated data (e.g., metrics). For example, in different embodiments, the system 202 can request and/or receive multiple brainwave data records. For example, in some embodiments, a user can submit at least a first set of brainwave data corresponding to a reference or baseline (“reference data”) and a second set of brainwave data corresponding to a calibration or brain activity recorded during engagement by the user with an active meditation or brain training session. As a general matter, the reference data can refer to EEG data that is collected while the user is in an “eyes closed” or “rest” condition (reference session), and the calibration data can refer to EEG data that is collected during the user's selected meditation style and level of experience (training session or calibration session). In some embodiments, additional (non-guided) meditation brainwave data can be collected as part of the protocol recommendation process. Additional data for each user, such as their past scores (scoring history), app usage history, and archived user metrics (user historical data 248) can also be optionally stored in or accessed by user accounts database 242. In some embodiments, this information can be retrieved by a UI manager 264 in response to user requests for account data and/or a description of their past performance and efforts across different mediation styles/levels. In other words, the UI manager 264 can present a summary of the user's brain training sessions as filtered by the meditation (brain state) protocol that was selected, as well as the selected level of experience that was indicated.

In different embodiments, the analyzer module 230 can perform its various operations on multiple sets of brainwave data that are received for a user. For example, the analyzer module 230 can process both the reference data and the calibration data and generate two sets of outputs. In some embodiments, the depth scoring module 250 can receive these outputs of the analyzer module 230, which can be used in turn to perform a specific sequence of operations that enable the brainwave data to be scored and assessed for its adherence to the selected protocol (depth) based on composite data.

In one example, the user can be requested to provide a few to several minutes of reference data and two minutes of meditation type-specific and experience level-specific calibration/practice data. In other embodiments, the amount of time over which data is collected can vary based on the user's preferences, including shorter durations (e.g., one minute) and longer durations (200 seconds, 3 minutes, 5 minutes, 10 minutes, and 20 or more minutes).

As will be described in greater detail below, in different embodiments, the data harvested from the calibration session can be used to calculate depth scores for neurofeedback on a 1-1000 scale, by reference to the baseline data. In one embodiment, for each rule of the selected protocol, a Z-score calculation module (“Z-score calculator”) 252 can calculate the Z-score for a pre-selected time increment in the calibration session by a formula where each Z-score=reading (value) for a specific metric during the calibration session over the time increment—the average for that metric across the reference session. As noted earlier, in one example, the metrics can reflect one second of data. Thus, for two minutes of data analyzed, there would be 120 time increments, and an associated 120 metric sets (one set of metrics for each second of EEG data).

In some embodiments, a statistical processor 254 can determine, for each metric, a standard deviation (SD) for the calibration data based on the reference data. In one optional embodiment, the depth scoring module 250 can establish a score range to improve score accuracy, including a minimum score (e.g., at −3 SDs) and a maximum score (e.g., at +3 SDs). A weight selection module 256 can then access weights for the rule linked to the given metric by reference to a protocol repository 280. In different embodiments, the protocol repository 280 stores a wide range of protocols, each protocol directed to the promotion of a technique toward a particular type of brain state. In some embodiments, the protocol repository 280 stores each of the available protocols, including each protocol's rules and each rule's assigned weight, as described in the McDonough applications. In one example, protocols can be grouped into a related set of meditations, as discussed earlier. This is reflected in a first grouping 272 (labeled for reference as brain state techniques group A) including first protocol set 274 of one or more protocols and a second grouping 276 (labeled for reference as brain state techniques group B) including second protocol set 278 of one or more protocols. Each grouping will typically including a different set of protocols, though in some cases there may be some overlap. As noted in FIG. 1A, a user may initially be allowed to choose the larger meditative scope or style (i.e., a grouping from a number of groupings available for purposes of convenience) before moving toward a specific protocol selection. However, in other cases, the protocols may simply be stored as a single collection and can all be accessed when determining whether a depth score was better or worse for a given protocol.

Thus, the weight selection module 256 can access the protocols in the protocol repository 280 and in a substantially synchronous process or sequential process identify the weight needed to perform a scoring operation by a weighted Z-score calculation module (“weighted Z-score calculator”) 258 for each rule in that protocol. If the user initially chose one subset of the protocols (e.g., a meditation style), the weight selection module 256 can limit its access to only those protocols that were in that subset. In different embodiments, for each of the protocols (each comprising one or more rules) that are being considered for recommendation to the user, the weight selection module 256 will identify the rule weights needed for use by the weighted Z-score calculation module 258. In other words, in some embodiments, each Z-score that is calculated for a given metric can be adjusted based on the rule weight associated to that metric (for that protocol). Thus, for each Z-score, a weighted Z-score is calculated by multiplication of the Z-score by the weight. In a next operation, a composite weighted average Z-score calculation module (“composite weighted average Z-score calculator”) 260 receives the weighted Z-scores and calculates the composite average for all of the metrics relevant to that protocol (each of the rules) for each time increment. In some embodiments, an optional offset can be applied to each of the composite weighted average Z-scores by an offset and scaling module 262. In one example, the value of a first offset can be selected to bring the lowest of the composite weighted average Z-scores to zero, and the value of a second offset can be selected to bring the highest of the composite weighted average Z-scores to the desired maximum. In addition, in some embodiments, a scaling multiplier may be applied to allow for a clearer distribution of scores, on a scale that is readily interpretable by humans. For example, each of the composite weighted average Z-scores can be multiplied by a scaling multiplier such as 1000/(maximum composite z-score−minimum composite z-score) (e.g., for a depth score range of 1-1000). In other embodiments, the scaling multiplier can be greater or smaller depending on the scoring range desired for user feedback.

The output of the depth scoring module 250 is then a set of at least two depth scores (or composite weight average z-scores). For example, a first depth score for the user's brainwave data 210 can be generated in the context of a first protocol, which comprises a first set of rules, while a second depth score for the same brainwave data 210 can also be generated, in the context now of a second (different) protocol which comprises a second set of rules. Although both of these scores are being used to represent the performance rating for the same brainwave data, the scores differ because the same performance is being evaluated multiple times by reference to different grading criteria. This process is repeated for all of the protocols in the protocol repository 280, or a subset of protocols therein, to generate a depth score set 268 that includes a plurality of depth scores each rating the brainwave data for a different protocol.

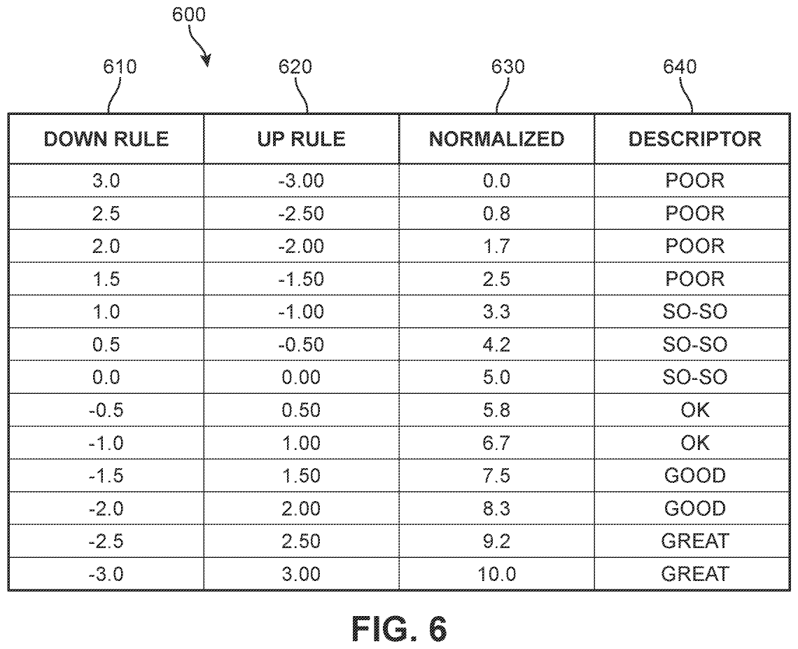

In different embodiments, each composite weighted average Z-score can then be used to rank the user's performance across multiple protocols. In one example, the depth scores 268—with one depth score for each protocol—can be shared with UI manager 264 for processing and representation via a visualization dashboard 284 that can be presented to the user. In some embodiments, a scoring feedback module 266 can prepare the scores for depiction in a time-series graph or bar graph or whisker plot or other graphic. Data about the protocols for which depth scores have been calculated can be accessed from protocol repository 280 via a protocol selector 288 and used to present additional insights and a detailed breakdown about the scores (e.g., see FIG. 6). Furthermore, a recommendation generator 286 can identify the highest depth score (or top 2-3 depth scores) and the corresponding protocol, which reflects the system's prediction of the protocol(s) that would be best suited to the user. The recommendation can also be presented to the user, in some cases with the option to immediately initiate a brain activity training session based on the top-rated protocol for that user. In one example, the system can automatically implement the brain activity training session based on the top-rated protocol for that user. In some embodiments, the recommendation can be stored in the user's account for their reference in subsequent sessions.

It should be understood that, as discussed in the McDonough applications, one or more of the protocols available in the protocol repository 280 can further comprise multiple sub-protocols, where each sub-protocol is directed to the same meditation technique at a different level of expertise. As some examples, there may be sub-protocols available, based on pre-designated segmented experience categories, such as: (a) Level 1—Beginner: <10 hours, (b) Level 2—Novice: 10-100 hours, (c) Level 3—Intermediate: 100-1,000 hours, (d) Level 4—Advanced: 1,000-10,000 hours, and (e) Level 5—Expert: >10,000 hours. However, ranges in each of these levels are presented as an example, and the category time/hour thresholds can be modified to allow for a more generalized meditation experience by reducing the number of levels, and the hours listed can be in some cases overlapping to accurately represent the fluid nature of meditation expertise and hours of practice for each neuropsychological profile. Furthermore, the sub-protocols can be modified to accommodate individuals who are able to move thorough these stages faster with talent, longer sessions, retreats, and various neurofeedback.

Thus, when the protocol recommendation system 220 accesses the weights to apply from the protocol repository 280, in some embodiments, the level of experience may impact which specific sub-protocols are selected for the depth score comparison. As one non-limiting example, the system 220 can be pre-configured to evaluate the user's performance with respect to all of the meditation techniques as prescribed by the weights for that technique at the “Level 2” experience. In another example, the brainwave data can be scored in the context of all of the sub-protocols (e.g., across all of the experience levels) to determine more specifically not only which meditation technique the user is best suited to but the experience level in that technique that is most appropriate.

The ability of the system to assess a user's brain activity against a set of protocols that take into account the level of experience of the persons providing the data is a vital aspect of the proposed systems. Most conventional meditation studies and their resultant data have been based on participants who were already expert meditators in order to capture brainwaves reflecting distinct altered states of consciousness. If the difference between a student's (beginner or novice) brain and an expert's brain was merely a matter for degree, the experience level might not be as pivotal—the system could be set up with the brain signature of only experts/masters as the “gold standard” and measure the student's depth of meditation by how closely the student's brain wave patterns approximate the brainwave patterns of the masters. However, the process of meditation—or the intentional progression toward any particular brain state—is far more complex in that the patterns of brainwaves that are dominant as the student develops skills and moves to mastery of the technique are not a simple linear progression. Instead, just as there are stages of learning for any complex instrument, students and masters will be engaged on different learning tasks. For example, in learning to play the violin: (a) the beginning violinist is generally focused on note production, and so they have to work hard to produce notes, not squeaks, (b) a novice generally works on tuning and timing (e.g., the C # needs to be a C # and the quarter note needs to be a quarter note), (c) an intermediate violinist may start working on vibrato and tone color, (d) an advanced violinist, who no longer has to think about tuning and time, can start working on expression (e.g., how do the notes flow into each other (or not) and how should the pace and volume go up and down to best express the musical intent), and (e) an expert may hardly think about technique, and instead focus on expressing a musical idea.

As a non-limiting example, in some contexts, beginner meditators are observed to produce greater alpha, while expert meditators produce very little alpha. In addition, advanced meditators produce more gamma in the back, but not the front, of the brain. Intermediate meditators and advanced meditators produce the most frontal theta, while beginner meditators and expert meditators produce less frontal theta than intermediate meditators. This may be due to theta corresponding to the brainwave of hard-working focus on taming the mind, and intermediate meditators work harder at taming the mind than beginner or expert meditators. These and general tendencies and patterns have been captured and defined by each sub-protocol so that there is fine-tuned guidance applicable to each of advanced, intermediate, novice, and beginning meditators.

With this context, the reader may appreciate that a person learning to meditate may also pass through different non-linear stages as they practice a particular technique. For example, a beginner meditator primarily focuses on sitting still and starts to work on quieting the mind, a novice meditator primarily focuses on quieting the mind, an intermediate meditator primarily focuses on honing their skills at maintaining focus, clarity, and equanimity, an advanced meditator has developed their capacity to focus so well that they can focus with ease and so can primarily engage in examining reality with greater detail and clarity (e.g., see the gaps in the perceptions that are usually glossed over), and an expert meditator can use their prodigious focusing skill to dismantle the ego and find oneness. Each of these levels of the same meditation style can thereby be linked to brainwave patterns that are not necessarily “more of the same”. The capacity of the proposed systems to select and recommend protocols that reflect this reality is thus essential to successful meditation practice.

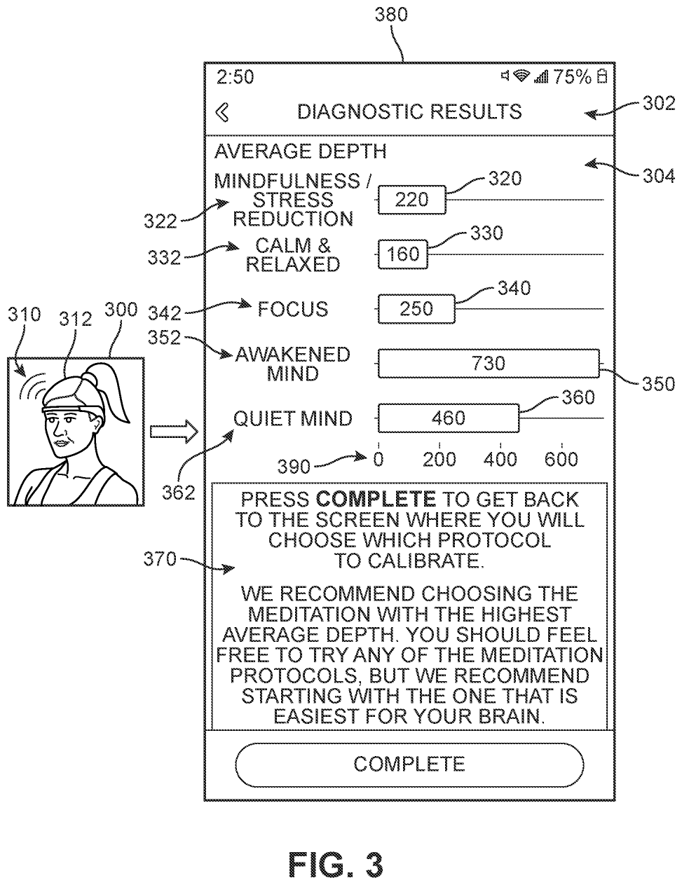

Referring briefly to FIG. 3, one example of a user interface including depth score graph 300 that may be presented to the user via their meditation application 302 following the operations described above is shown for purposes of illustration. In this example, a second user 312 in a meditative condition or state is shown transmitting her brainwave data 310 to a meditation application 302 during a brain data collection session 330. For example, the user may have entered a general meditative condition (their ‘usual’ style) for two minutes, during which their brain activity was recorded. The second user 312 can be understood to have selected modern meditations, followed by a request to implement the protocol recommendation service (help me choose option). In this case, the modern meditations include five meditation techniques. These five techniques are listed along the Y-axis of the depth score graph 300, and include a first protocol 322 (labeled here as mindfulness/stress reduction), a second protocol 332 (labeled here as calm and relaxed), a third protocol 342 (labeled here as focus), a fourth protocol 352 (labeled here as awakened mind), and a fifth protocol 462 (labeled here as quiet mind). Once the brain data collection session 330 is completed, the protocol recommendation system associated with the meditation application 302 can evaluate the brainwave data 310 across each of the protocols included in the modern meditations and generate depth scores reflecting the user's performance with respect to each. In other embodiments, the specific techniques (protocols) that are used to evaluate the brainwave data can vary.

In this example, each protocol listed includes a depth score that was generated by the protocol recommendation system described herein. Thus, the first protocol 322 is associated with a first depth score 320 (e.g., “220”), the second protocol 332 is associated with a second depth score 330 (e.g., “160”), the third protocol 342 is associated with a third depth score 340 (e.g., “250”), the fourth protocol 352 is associated with a fourth depth score 350 (e.g., “730”), and the fifth protocol 362 is associated with a fifth depth score 360 (e.g., “460”). The depth scores are graphically represented as a series of bars of the bar graph to express the relative magnitude of the scores. Thus, it becomes obvious that the highest (best) depth score corresponds to the fourth depth score 350, and the lowest (worst) depth score corresponds to the second depth score 330. In some embodiments, the protocol recommendation system can also rank the depth scores and present them in order from lowest to highest for the user.

Based on this information, the system can determine the current user has the greatest likelihood of succeeding in her meditation endeavor by selection of the fourth protocol 352 (in this case, awakened mind). In response to this analysis, the system can prompt the user to initiate a brain training session. In one embodiment, an option can be presented with the graph that, if selected, causes the system to automatically implement a meditative training session based on the recommended protocol. It can be appreciated that a person's brainwave data can change over time, especially as they become more adept at one or more meditation practices, and so brainwave data collected at a later date may lead to a different set of depth scores that would prompt the system to recommend a different protocol.

In other embodiments, the data is more comprehensively analyzed across all of the sub-protocols (i.e., across all of the available experience levels) to determine the optimal approach for the user. For example, the system can be configured to authorize access to additional information to the user and/or another authorized person who may participate in the user's meditation teaching as a coach. In such cases, the depth score graph 300 may be visualized instead by a more nuanced whisker plot that can display the average depth scores for all mediation groups and corresponding protocols across all experience levels. In one example, a coach can access a series of multiple (e.g., over 100) horizontal whisker plots sorted by average depth scores with a row for each protocol and experience level. In one embodiment, the user interface offers options for the whisker plots to also be sorted by meditation style/group and experience level.

Returning to FIGS. 2A and 2B, in different embodiments, the depth scoring process can be performed by the system 220 in real- or near-real-time as the brainwave data 210 is being received. In other embodiments, the process can be performed after the initial open meditative brainwave data is received. In one embodiment, the process is performed locally on the user's device via app 204. In another embodiment, some or all operations of the process are performed remotely and results are returned to the user's device over a network connection.

It should be understood that, in other implementations, environment 200 can include additional or fewer modules or can include one or more additional computing devices or related server devices. The modules of environment 200 can be associated with the various local computing devices and, for example, can be disposed within the computing device. In alternative implementations, the modules of environment 200 can include independent computing devices that are coupled to, and in data communication with, the local computing devices. As used in this description, the term “module” is intended to include, but is not limited to, one or more computers, processing units, or devices configured to execute one or more software programs that include program code that causes a processing device(s) or unit(s) of the computer to execute one or more functions. Processing units can include one or more processors (e.g., microprocessors or central processing units (CPUs)), graphics processing units (GPUs), application specific integrated circuits (ASICs), or a combination of different processors. In alternative embodiments, systems and modules can each include other computing resources/devices (e.g., cloud-based servers) that provide additional processing options for performing one or more of the machine learning determinations and calculations. The processing units or devices can further include one or more memory units or memory banks. In some implementations, the processing units execute programmed instructions stored in memory to cause system, devices, and modules to perform one or more functions described herein. The memory units/banks can include one or more non-transitory machine-readable storage mediums. The non-transitory machine-readable storage medium can include solid-state memory, magnetic disk, and optical disk, a random-access memory (RAM), a read-only memory (ROM), an erasable programmable read-only memory (e.g., EPROM, EEPROM, or Flash memory), or any other tangible medium capable of storing information.

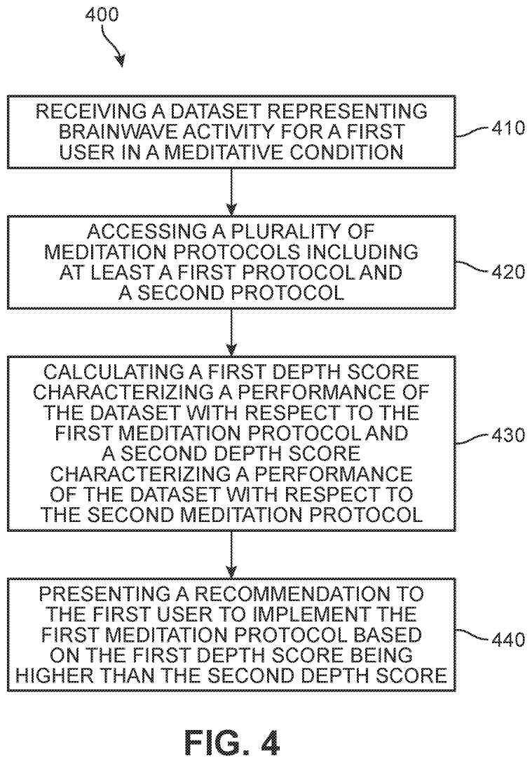

FIG. 4 is a flow chart illustrating an embodiment of a method 400 for automatically identifying and generating personalized brain state protocol recommendations. The method 400 can include a first step 410 of receiving, at a protocol recommendation system for a meditation application, a dataset representing brainwave activity for a first user in a meditative condition, and a second step 420 of automatically accessing, at the protocol recommendation system, a plurality of meditation protocols including at least a first meditation protocol and a second meditation protocol, where each meditation protocol promotes a different type of meditation experience. In one embodiment, the operation of accessing the protocols can occur in response to receiving the dataset. In addition, the method 400 can include a third step 430 of calculating, by the protocol recommendation system, at least a first depth score characterizing a performance of the dataset with respect to the first meditation protocol and a second depth score characterizing a performance of the dataset with respect to the second meditation protocol, where the first depth score is higher than the second depth score. A fourth step 440 includes presenting, via a user interface for the meditation application and in response to the first depth score being higher than the second depth score, a recommendation to the first user to implement the first meditation protocol.

In other embodiments, this method may include additional steps or aspects. In one example, the method 400 also includes steps of receiving, from the first user and via the user interface, a selection of the recommendation; and providing, via the meditation application, a first guided meditation session that is implemented based on the first meditation protocol. In another example, the method 400 includes steps of automatically presenting, via the meditation application and prior to presenting the recommendation, a first list of options including a first option representing the first meditation protocol and a second option representing the second meditation protocol; automatically presenting, via the meditation application, a third option for predicting a user's optimal meditation protocol; receiving, via the user interface and from the first user, a selection of the third option; and automatically initiating, via the meditation application and in response to the selection of the third option, a first brain activity collection session during which the dataset is obtained. In some cases, additional steps can also include presenting, via the user interface and prior to presenting the first list of options, a second list of options including a fourth option representing a first meditation style and a fifth option representing a second meditation style. In such cases, the first meditation style can include both the first meditation protocol and the second meditation protocol, and the second meditation style can include a third meditation protocol. In addition, the method can include receiving, via the user interface and from the first user, a selection of the fourth option, where presentation of the first list of options is in response to the selection of the fourth option.

In some embodiments where the first meditation style includes meditation protocols for different user experience levels, the method can also include steps of calculating depth scores for each of the meditation protocols of the first meditation style; generating a first graph depicting the depth scores, the first graph indicating to which protocol and experience level each depth score corresponds; and presenting the first graph to a second user who is authorized to access additional data regarding the first user for purposes of coaching. In another embodiment where the plurality of meditation protocols includes meditation protocols that each promote a different type of meditation experience for the same experience level, the method can also include calculating depth scores for each of the meditation protocols in the plurality of meditation protocols; generating a first graph depicting all of the calculated depth scores; and presenting, via the meditation application, the first graph to the first user. In one example, the first depth score is the highest depth score displayed on the first graph. In some embodiments, each depth score visually depicted on the first graph also corresponds to a directly selectable option to initiate a guided meditation session based on the meditation protocol associated with that score such that the first graph also serves as an interactive menu.

In different embodiments where the first meditation protocol includes at least a first rule and a second rule, the method can also include receiving, at the protocol recommendation system, a guided meditation dataset representing brainwave activity for the first user during the first guided meditation session, calculating, by the protocol recommendation system, a first score characterizing a performance of the guided meditation dataset with respect to the first rule and a second score characterizing a performance of the guided meditation dataset with respect to the second rule; presenting, via the meditation application, both the first score and the second score; receiving, via the user interface, a request to improve the first score; and automatically creating, at the meditation application and in response to the request, a second meditation protocol including only the first rule. In yet another embodiment where the first meditation protocol includes at least a first rule and a second rule, the method can also include receiving, at the protocol recommendation system, a guided meditation dataset representing brainwave activity for the first user during the first guided meditation session; calculating, by the protocol recommendation system, a first score characterizing a performance of the guided meditation dataset with respect to the first rule and a second score characterizing a performance of the guided meditation dataset with respect to the second rule; and presenting, via the meditation application, a dashboard including: a description of the first rule accompanied by the first score, and a description of the second rule accompanied by the second score.

In different embodiments, a system for generating personalized brain state protocol recommendations is disclosed. The system includes a processor and machine-readable media including instructions which, when executed by the processor, cause the processor to perform some or all of the steps described above.

As a general matter, it may be appreciated that a target brain state may be more effectively and efficiently achieved when feedback is accurate and detailed. In other words, as a person is working toward a specific brain state, they can more readily pinpoint the appropriate ‘zone’ or pattern of brain activity when they receive timely and easy-to-comprehend feedback about their performance, and that feedback is based on a set of parameters (e.g., protocol) that have been selected for facilitating that specified brain state at the person's experience level. As will be described below, a reliable and comprehensive feedback paradigm can allow each user to quickly discern and absorb how their performance measured against their goals. This knowledge can then be applied to modify their practice. Furthermore, in some embodiments, the ability to monitor their meditation performance not just as a sequence of high-level depth scores but in a more detailed breakdown can fundamentally shift how a person approaches their meditation practice.

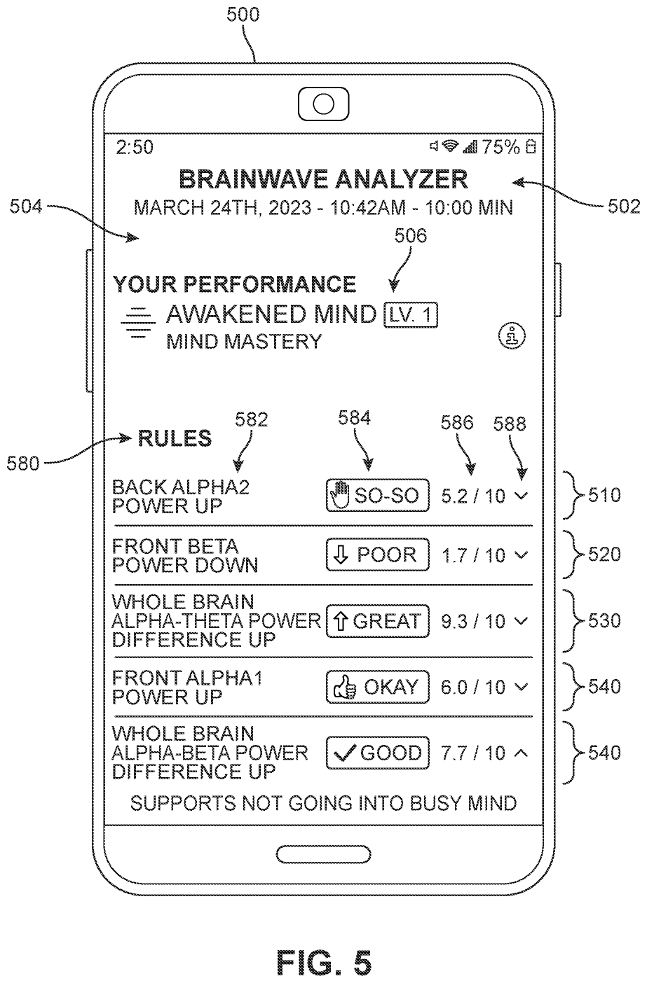

For purposes of introduction to an embodiment of a brain state feedback system, an example of a user feedback dashboard interface (“dashboard”) 504 is shown in FIG. 5 as presented via a meditation application (“app”) 502 running on a mobile computing device 500. In this example, the user has completed a brain activity training session based on one of the protocols available in the protocol repository. For purposes of this example, the user had selected a meditation subprotocol 506 here identified as “Awakened Mind/Mind Mastery” for Experience Level 1. Thus, a brainwave data set has been received by the app 502 reflecting the user's practice during the session. The system has processed the brainwave data as described in the McDonough applications and FIGS. 2A and 2B above and measured the user's performance in the scope of the selected technique that was implemented during the session. It should be understood that the brain state feedback system can include some or all of the features, modules, components, and functions provided by the protocol recommendation system described earlier.

More specifically, the brain state feedback system can access each of the individual rules in the meditation subprotocol 506 and, for each of these rules, calculate the user's overall change from the base state for that rule. In other words, since each rule is created based on a specific metric that was found to be significant for that meditation technique and experience level, the user's brainwave activity can be assessed by determining how close (or far) the user's EEG was to the base state defined by that rule. Thus, a user's performance is no longer simply a comprehensive depth score, but it can be broken down into grades describing their performance in different aspects or features of the broader technique. These calculations can, for example, be performed by the statistical processor. In one example, the changes from the base state—for each rule—are measured in standard deviations. In some embodiments, the system can then translate or convert each standard deviation to a rating or score that falls somewhere along a pre-set range (e.g., 0-10, 0-100, etc.). Furthermore, in some embodiments, the brain state feedback system can generate a text-based descriptor to accompany each rating that can serve as a more user-friendly appraisal of the user's performance with respect to each rule.

In FIG. 5, the dashboard 504 presents one example of the outcome of this process. For example, for the meditation subprotocol 506, the system identified five rules. The user's brainwave data was then processed and evaluated with respect to each of these five rules to generate five distinct feedback components 580. In some embodiments, each of the feedback components 580 can include a label 582 that identifies the rule and its goal, a one-word/phrasing descriptor 584 offering immediate insight into the performance, a rating 586 indicating the user's relative performance level, and a drop-down option 588 to view additional details regarding the significance of the selected rule and the user's performance. As shown in FIG. 5, a first component 510 directed to a “back alpha2 power up” rule was described as “so-so” and rated 5.2/10, a second component 520 directed to a “front beta power down” rule was described as “poor” and rated 1.7/10, a third component 530 directed to a “whole brain alpha-theta power difference up” rule was described as “great” and rated 9.3/10, a fourth component 540 directed to a “front alpha1 power up” rule was described as “okay” and rated 6.0/10, and a fifth component 550 directed to a “whole brain alpha-beta power difference up” rule was described as “good” and rated 7.7/10.

In different embodiments, the brain state feedback system can include a feedback database that stores the individual feedback output that should be provided in response to each of level of performance. One example of a look-up table 600 that may be accessed by or included in the system is depicted in FIG. 6. It is to be understood that the values shown in the table 600 are included for illustrative purposes, and the ranges and descriptors can be adjusted as desired to best accommodate the goals of the practice. The table 600 represents possible feedback that will be generated in response to the standard deviation that is calculated for the brainwave data relative to a baseline associated with that rule. Thus, a first column (“down rule”) 610 is directed to metrics where a more negative standard deviation (<0 to −3) from the baseline is better or more aligned with the rule's target brain activity, and a more positive standard deviation (0 to +3) is worse or farther from the rule's target brain activity. A second column (“up rule”) 620 is directed to those metrics where a more negative standard deviation (0 to −3) from the baseline is worse or farther from the rule's target brain activity, and a more positive standard deviation (>0 to +3) is better or more aligned with the rule's target brain activity. For each of these possible standard deviations, as calculated by the statistical process for the brain state feedback system, a normalized score can also be generated, which is shown in a third column (“normalized”) 630, that converts the standard deviations to a uniform rating that falls within a predesignated minimum (e.g., zero) to a predesignated maximum (e.g., 10.0). Finally, in a fourth column (“descriptor”) a text-based translation is stored that describes in language a summarized feedback that can be quickly understood by the user (e.g., ranging from poor, so-so, okay, good, to great). These terms can be changed depending on the user's language and preferred expressions. In addition, in some embodiments, greater discrimination can also be provided, such that each of the 8 rows or rating levels can be assigned its own unique descriptor. In other embodiments, the standard deviations can be further divided into smaller increments (<0.5) to allow for more levels of feedback to be defined, or larger increments (>0.5) to allow for fewer levels of feedback to be defined. In other words, the feedback paradigm is readily customizable.

In different embodiments, the brain state feedback system can also include a strategy repository that identifies tailored recommendations for supporting a user in their goals. In some embodiments, the strategy repository can store, for a given rule in a meditation protocol, a specific action that the user can incorporate into their meditation that is known to improve a person's performance with respect to the rule. Some non-limiting examples of such a repository are presented in Table 1 below for the meditation directed to an “Awakened Mind” protocol. It is to be understood that the fields shown in Table 1 are included for illustrative purposes, and the descriptors can be adjusted as desired to best accommodate the goals of the practice.

TABLE 1

| | | Freq | Freq | | | | |
|---|---|---|---|---|---|---|---|
| Location | Band | Low | High | Measurement | Direction | Rule Name | Strategy |
| Back | Delta | 2 | 4 | Power | Up | Back Delta | Try using Delta-frequency |
| | | | | | | Power Up | binaural beats during the |
| | | | | | | | session |
| Front | Theta2 | 6.25 | 8 | Power | Up | Front | Try visualizing creative |
| | | | | | | Theta2 | ideas and solutions during |
| | | | | | | Power Up | meditation |
| Front | Alpha1 | 8 | 10 | Power | Up | Front | Try slow, deep breathing |
| | | | | | | Alpha1 | to induce relaxation |
| | | | | | | Power Up | |

In different embodiments, as each rule is scored as described above and the dashboard of generated, a user can be offered not just feedback about their performance, but rule-specific guidance (tips) that can be incorporated into their practice to help them improve. In the context of the example shown in FIG. 5, a user who realizes, upon review of the dashboard 504, that their “Front Alpha1 Power Up” rule performance as average or “OKAY” may be motivated to raise their overall meditation by incorporating actions that are known to raise the component of their performance that is directed to the Front Alpha1 Power Up rule. The dashboard 504 can then offer the user “tips” for improving their eliciting of brainwave activity in specific components.

In this case, the dashboard 504 could further present a message to “Try slow, deep breathing to induce relaxation” in same section as the scoring of the fourth component 540, noting that this action is specifically known to improve users' ability to perform with respect to Front Alpha1 Power Up. In some embodiments, such a tailored recommended action or strategy can be presented in each of the feedback component scoring sections. In other words, for first component 510, a first strategy directed to improving the user's performance with respect to the first rule listed (e.g., “Back Alpha2 Power Up”) will be presented adjacent to the first rule's score, for second component 520, a second strategy different than the first strategy directed to improving the user's performance with respect to the second rule listed (e.g., “Front Beta Power Down”) will be presented adjacent to the second rule's score, for third component 530, a third strategy different both the first strategy and the second strategy directed to improving the user's performance with respect to the third rule listed (e.g., “Whole Brain Alpha-Theta Power Difference Up”) will be presented adjacent to the third rule's score, and so forth for each rule in the protocol. In some embodiments, the user can select the “expand” or drop-down option to reveal more information about their score, and this additional information can include the related actionable strategy that can improve the user's performance with respect to the selected rule.

As described herein, each actionable strategy is directly tuned to improvement of a user in developing their skills for brainwave activity of a specific rule. In other words, the actionable strategy selected and then shown to the user in each instance is not a generic or broad prescription but is crafted to supporting and promoting a user's performance in a narrow subset (rule) of the overall meditation protocol. In some embodiments, each actionable strategy in the strategy repository is based on at least one peer-reviewed brainwave study or other robust neuroscience research in which the strategy was shown to promote/elicit the desired brainwave activity. In different embodiments, each actionable strategy can refer to a specific physical touchstone, process, or awareness and/or a mental strategy, internal focus, or activity that will allow the user to intentionally move toward an improvement in their score for the selected rule.

In addition, in some embodiments, these suggested actions, each tailored to improving performance of brain activity with respect to a specific rule, can also be re-presented at subsequent practice sessions where the user continues the same meditation protocol. In other words, when the user later returns to the app and selects an option to continue to practice the same protocol (e.g., Awakened Mind), the system can be configured to remind the user (e.g., via an in-app notification or message) of a selected strategy linked to one of their weaker performing components (rules). In some embodiments, the user can be asked if they want the app to present such reminders that are based on their past performance, in future sessions. Thus, while viewing the feedback components listed in the dashboard 504, a user could select one or two or more rules they want to improve, and a request that the app remind them of the rule-specific strategies that could improve their performance in the upcoming session when they are about to start that session. In response to this selection, the app will automatically present the specific strategy to the user when the re-open the same meditation protocol to begin the next practice session. For example, if the user previously indicated their desire to improve their performance in their “Front Alpha1 PowerUp” component when they next meditate with the “Awakened Mind” protocol, the app will automatically present a reminder message showing the actionable strategy tailored to facilitate Front Alpha1 PowerUp that the user can incorporate into their session at the time the session is being opened or loaded.

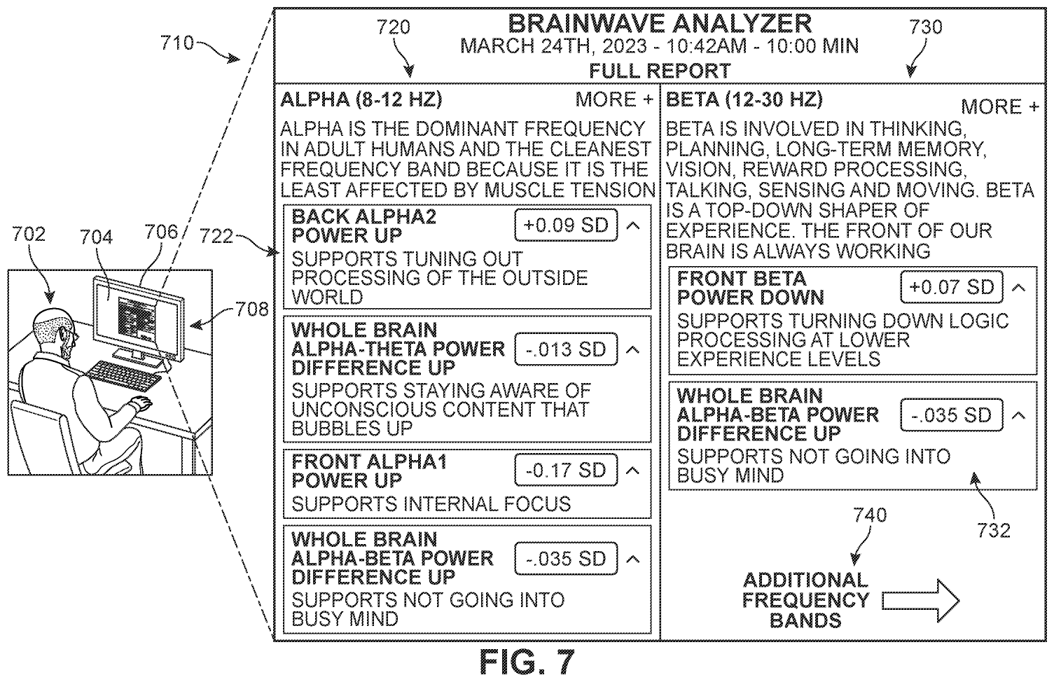

Moving now to FIG. 7, another example of feedback that may be generated by the brain state feedback system is depicted. In FIG. 7, a more detailed or comprehensive analysis is offered and presented to a third user 702 following a brain activity training session. The brainwave data submitted by the third user 702 has been processed and is provided to the third user 702 as a report 710. The report 710 can be accessed via the app itself, or can be provided to the user via text or website link or e-mail for their later perusal if they prefer a larger display 706 of a desktop computer 708 for example. In this case, the third user 702 is accessing their email application 704 and opened an email in which the system-generated report 710 has been included. In different embodiments, the report 710 can include details described earlier with respect to dashboard 504 in FIG. 5, such as the protocol and the rule identifier labels. However, report 710 also includes information about each rule to facilitate the user's understanding of the data. In some embodiments, the brain state feedback system includes an interpretation database that includes descriptive paragraphs that interpret the scoring calculated for each rule. In one example, a report writer module of the brain state feedback system can pull interpretation paragraphs from the interpretation database and automatically generate a report that includes interpretations of increases and decreases for each metric in the meditation protocols. In some embodiments, the report can also include a Frequency Band Power Interpretive Report that interprets the meaning of all changes from baseline for each of the frequency bands. In one example, the report interprets those changes that reflect a shift of at least some minimum pre-designated increment of the standard deviation (e.g., 0.25, 0.5, 1, etc.) from the baseline for a given frequency band. This increment can be preselected and set as a default by the system or can be user-modifiable based on the user's desired shift increment. The report 710 can then offer interpretations of the changes across multiple brain areas, such as for the whole brain, front vs. back, and left vs. right, and organized across each of the five frequency bands (e.g., alpha band 720 and beta band 730, etc.), and the rules that have some overlap with those bands (e.g., alpha-related rules 722 and beta-related rules 732, etc.). The interpretation paragraphs can be accessed by the system and presented in the corresponding slot for the selected rule to explain what the increase or decrease for each frequency band in each location actually means for the user's experience and practice.

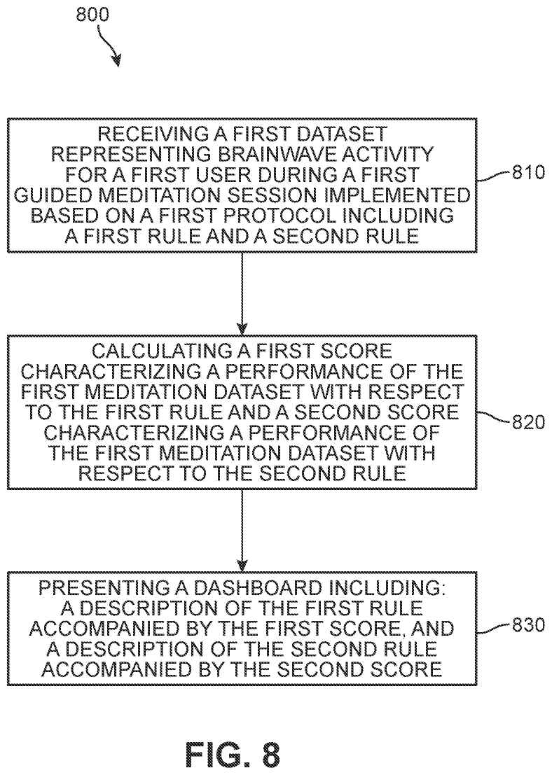

FIG. 8 is a flow chart illustrating an embodiment of a method 800 for providing personalized meditation feedback. The method 800 can include a first step 810 of receiving, at a brainwave feedback system for a meditation application, a first dataset representing brainwave activity for a first user during a first guided meditation session implemented based on a first meditation protocol, the first meditation protocol including at least a first rule and a second rule. A second step 820 includes calculating, by the brainwave feedback system, a first score characterizing a performance of the first meditation dataset with respect to the first rule and a second score characterizing a performance of the first meditation dataset with respect to the second rule. In addition, the method 800 can include a third step 830 of presenting, via the meditation application, a dashboard including: a description of the first rule accompanied by the first score, and a description of the second rule accompanied by the second score.

In other embodiments, this method may include additional steps or aspects. In one example, the dashboard further includes a first one word or two word descriptor or phrase (text/language based) accompanying the first score and a second one word or two word descriptor or phrase (text/language based) accompanying the second score. In another example, the first rule is directed to brain activity promoting a first metric and the second rule is directed to brain activity promoting a different, second metric. In some embodiments, the method 800 also includes accessing, at the meditation application, a frequency band power database that includes a first set of text-based descriptions interpreting a range of scores possible for the first metric, and a second set of text-based descriptions interpreting a range of scores possible for the second metric; selecting a first text-based description linked to the first score from the first set and selecting a second text-based description linked to the second score from the second set; and presenting, via the meditation application, both the first text-based description and the second text-based description. In other embodiments, the method 800 can further include receiving, via a user interface for the meditation application, a first request to improve the first score; automatically creating, at the meditation application and in response to the first request, a second meditation protocol including only the first rule; and initiating, at the meditation application, a second guided meditation session for the first user that is implemented based on the second meditation protocol.

In some embodiments, a system for providing personalized meditation feedback is disclosed. The system includes a processor and machine-readable media including instructions which, when executed by the processor, cause the processor to perform some or all of the steps described above.

In different embodiments, the proposed systems can include provisions for creating and executing user-tailored brain exercise programs based on the data included in the detailed breakdown presented by the brain state feedback system. In other words, while a person visiting a gym to ‘workout’ may have a larger goal of becoming a better swimmer, they may approach the goal not simply or only by actually swimming laps, but by carefully selecting individual exercises that help them address the areas of their physique that would confer an advantage during their swims. For example, they may opt to engage in cardio exercises such as running to improve their stamina, or lift weights to improve their arm strength. Thus, a person can focus on discrete aspects of the larger target in order to improve their practice. In particular, they may choose to strengthen in areas that they have seen contribute to a less than optimal performance.

In different embodiments, a remedial protocol creation tool incorporated by the brain state feedback system can offer meditation practitioners the opportunity to focus on elements of their meditation which they understand or believe they might develop to further their selected holistic meditative approach. In one example, a user might review their scores for a given brainwave activity training session as described above (e.g., see FIGS. 5, 6, and 7). The feedback can indicate their relative strengths and weaknesses in terms of the specific rules associated with their selected protocol. This information can then be used by the remedial protocol creation tool to create a personalized meditation experience (workout) that targets only those brainwave patterns selected by the user.

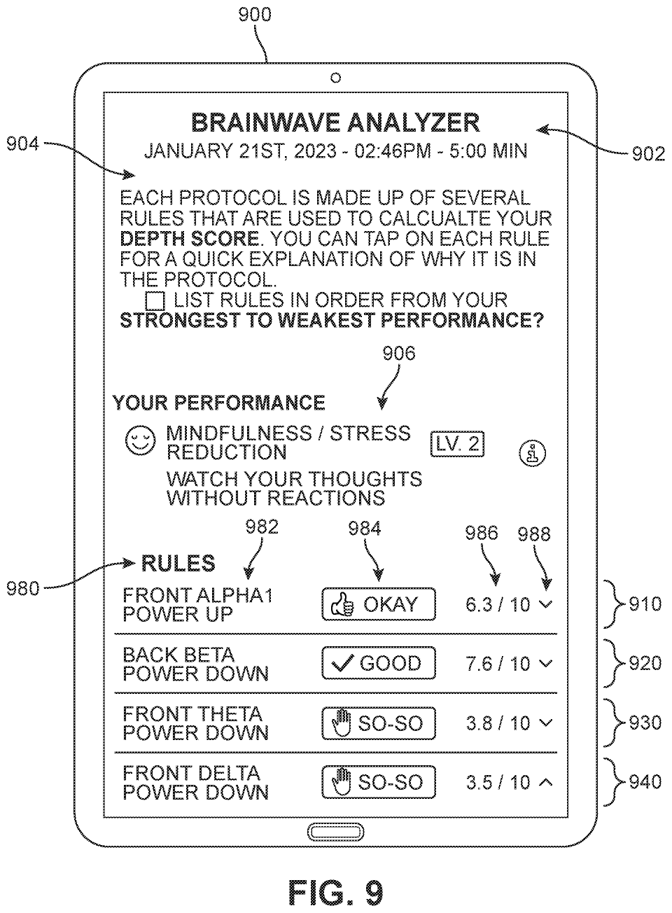

For purposes of illustration, a sequence of drawings in FIGS. 9, 10, 11, and 12 depicts one example of such a process. In FIG. 9, another example of a user feedback dashboard interface (“dashboard”) 904 is shown as provided via a meditation application (“app”) 902 running on a mobile computing device 900. In this example, a user has again completed a brain activity training session based on one of the protocols available in the protocol repository. For purposes of this example, the user had selected a meditation subprotocol 906 here identified as “Mindfulness/Stress Reduction” for Experience Level 2. Thus, a brainwave data set has been received by the app 902 reflecting the user's practice (with the goal of achieving a brain state associated with the mindfulness meditation technique) during the session. The system has processed the brainwave data as described in the McDonough applications and FIGS. 2A and 2B above and measured the user's performance in the scope of the selected technique that was implemented during the session.

More specifically, the brain state feedback system can access each of the individual rules in the meditation subprotocol 906 and, for each of these rules, calculate the user's overall change from the base state for that rule. In other words, since each rule is created based on a specific metric that was found to be significant for that meditation technique and experience level, the user's brainwave activity can be assessed by determining how close (or far) the user's EEG was to the base state defined by that rule. In FIG. 9, the dashboard 904 presents one example of the outcome of this process. For example, for the meditation subprotocol 906, the system recognized four rules. The user's brainwave data was then processed and evaluated with respect to each of these four rules to generate four distinct feedback components 980. As noted earlier, in some embodiments, each of the feedback components 980 can include a label 982 that identifies the rule and its goal, a language-based term such as a one-word/phrasing descriptor 984 offering immediate insight into the performance, a rating 986 indicating the user's relative performance level, and a drop-down option 988 to view additional details regarding the significance of the selected rule and the user's performance. As shown in FIG. 9, a first component 910 directed to a “front alpha1 power up” rule was described as “okay” and rated 6.3/10, a second component 920 directed to a “back beta power down” rule was described as “good” and rated 7.6/10, a third component 930 directed to a “front theta power down” rule was described as “so-so” and rated 3.8/10, and a fourth component 940 directed to a “front delta power down” rule was also described as “so-so” and rated 3.5/10.

The user in this case may recognize that while they appear to be doing quite well with respect to one facet of the technique (first component 910), and reasonably well with respect to a second facet of the same technique (second component 920), their performance with respect to the remaining two facets (third component 930 and fourth component 940) leave something to be desired. In some embodiments, the user could simply continue to take part in training sessions based on the same protocol, with the hope that they will improve in these areas of weakness along with their overall depth score as they practice. However, in other embodiments, the user may opt to selectively target those aspects of their technique in which they seem to be performing below their goal.

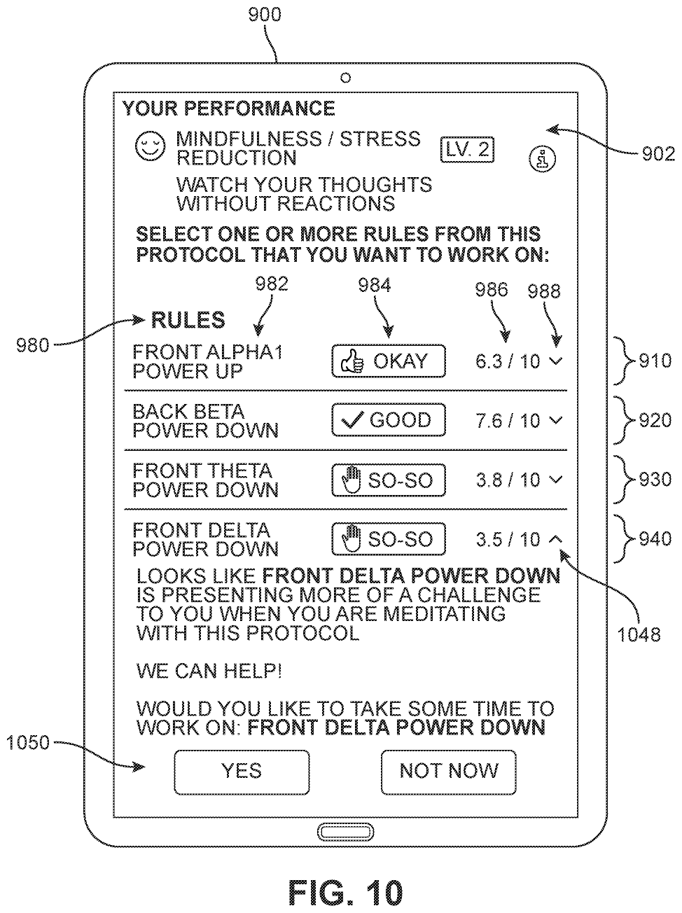

As shown in FIG. 10, the user selects a first drop-down option 1048 for the fourth component 940, causing additional information and options to be shown. In this case, a remedial protocol message 1050 is provided, in which the selected rule is identified (e.g., “Looks like FRONT DELTA POWER DOWN is presenting more of a challenge to you when you are practicing this meditation. We can help!”) along with an offer to create a new protocol (e.g., “Would you like to take some time to work on FRONT DELTA POWER DOWN?”) that can be shown with options such as YES, NOT NOW, NO, or return to previous menu, etc. For purposes of this example, the user may select YES.

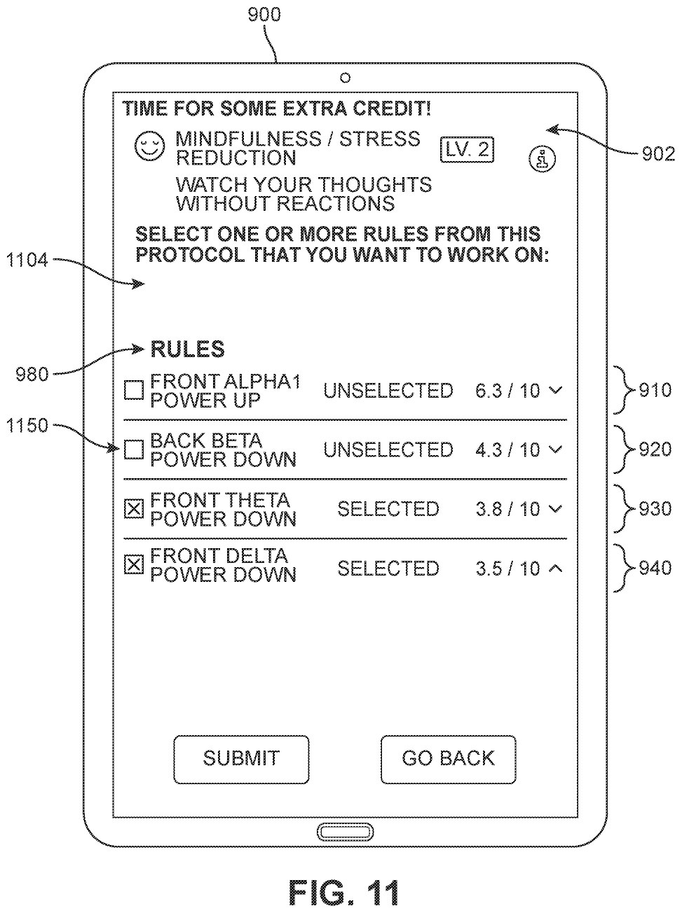

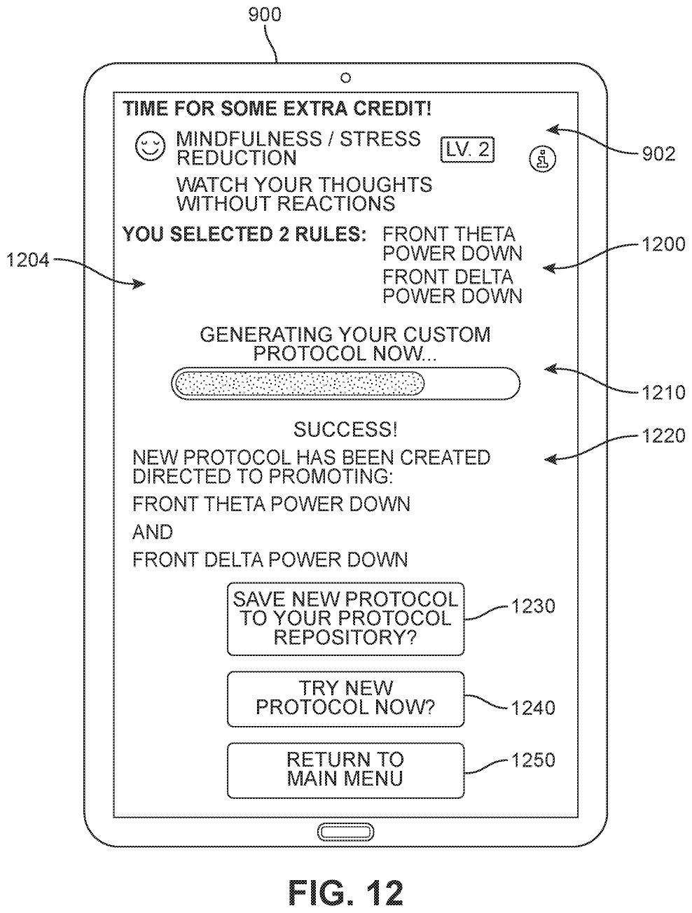

In different embodiments, in response to the user's agreement, the system presents a first remedial protocol creation user interface 1104 that serves as a front-facing end of the remedial protocol creation tool, as shown in FIG. 11. In this example, the feedback components 980 are again listed, and accompanied by a set of selectable options 1150. In other words, the app 902 can allow the user to select a subset of the full protocol at this stage. For purposes of this application, a subset of a protocol (or “child protocol”) refers to a number of rules that is less than the complete number of rules for that protocol (i.e., at least one less rule), where the total number of rules for the full protocol (or “parent protocol”) is at least two. In some cases, the user may only select one component. In other cases, the user can select more than one component, but less than all of the listed components for the protocol. In FIG. 11, the user selects their two weakest performing components (third component 930 and fourth component 940). They can confirm their selection, and this subset 1200 is presented for review in a second remedial protocol creation user interface 1204, an example of which is shown in FIG. 12. In some embodiments, the system can initiate a protocol creation cycle 1210 that incorporates only the selected subset 1200 of rules. Once the protocol has been created, the system can present a message 1220 to the user confirming the availability of the protocol, along with one or more options. In some embodiments, the protocol can be automatically added to the user's protocol repository for their use. In another embodiment, a first option 1230 can allow the user to decide whether the protocol should be saved. In some embodiments, the protocol can be automatically launched and implemented in a brain activity training session. In another embodiment, a second option 1240 can allow the user to decide whether to move next into the brain activity training session based on this new protocol. In another example, the user may simply return to the main menu (e.g., a third option 1250) or otherwise navigate elsewhere.

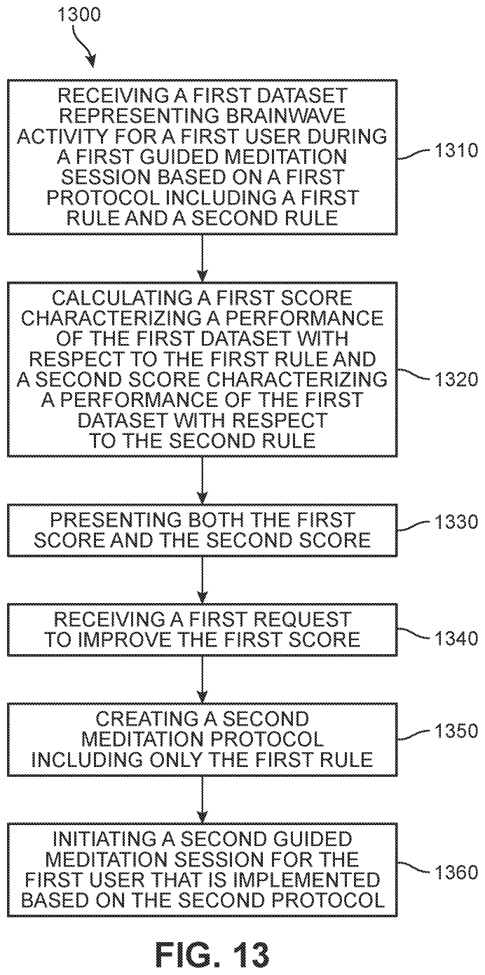

FIG. 13 is a flow chart illustrating an embodiment of a method 1300 for providing personalized brain state guidance. The method 1300 can include a first step 1310 of receiving, at a brainwave guidance system for a meditation application, a first dataset representing brainwave activity for a first user during a first guided meditation session implemented based on a first meditation protocol, the first meditation protocol including at least a first rule and a second rule, and a second step 1320 of calculating, by the brainwave guidance system, a first score characterizing a performance of the first dataset with respect to the first rule and a second score characterizing a performance of the first dataset with respect to the second rule. In addition, the method 1300 can include a third step 1330 of presenting, via the meditation application, both the first score and the second score, and a fourth step 1340 of receiving, via a user interface for the meditation application, a first request to improve the first score. The method 1300 can further include a fifth step 1350 of automatically creating, at the meditation application and in response to the first request, a second meditation protocol including only the first rule, and a sixth step 1360 of initiating, at the meditation application, a second guided meditation session for the first user that is implemented based on the second meditation protocol.

In other embodiments, this method may include additional steps or aspects. In one example, the method 1300 also includes determining, at the meditation application, the first score falls below a predesignated threshold; and presenting to the first user, in response to the first score falling below the predesignated threshold, a message recommending the creation of the second meditation protocol in order to improve their performance for the first meditation protocol. In one embodiment, the first rule is directed to brain activity promoting a first metric and the second rule is directed to brain activity promoting a different, second metric. In different embodiments, the first metric represents one of power, percent of total power, power ratio, coherence, connectivity, minimum frequency, maximum frequency, phase synchrony, complexity, brain location, and target brainwave direction.

In different embodiments, the method 1300 can also include steps of receiving, at the brainwave guidance system, a second dataset representing brainwave activity for the first user during a second guided meditation session implemented based on a third meditation protocol, the third meditation protocol including at least a third rule, fourth rule, and a fifth rule; calculating, by the brainwave guidance system, a third score characterizing a performance of the second dataset with respect to the third rule, a fourth score characterizing a performance of the second dataset with respect to the fourth rule, and a fifth score characterizing a performance of the second dataset with respect to the fifth rule; presenting, via the meditation application, the third score, fourth score, and the fifth score; receiving, via the user interface, a second request to improve both the fourth score and the fifth score; and automatically creating, at the meditation application and in response to the second request, a fourth meditation protocol including only the fourth rule and the fifth rule.

In another example, the method 1300 can further include steps of receiving, at the brainwave guidance system, a second dataset representing brainwave activity for the first user during a second guided meditation session implemented based on a third meditation protocol, the third meditation protocol including at least a third rule, fourth rule, and a fifth rule; calculating, by the brainwave guidance system, a third score characterizing a performance of the second dataset with respect to the third rule, a fourth score characterizing a performance of the second dataset with respect to the fourth rule, and a fifth score characterizing a performance of the second dataset with respect to the fifth rule; determining, at the meditation application, both the third score and the fourth score fall below a predesignated threshold, while the fifth score is above the predesignated threshold; and automatically creating, at the meditation application and in response to the third score and the fourth score falling below the predesignated threshold, a fourth meditation protocol including only the third rule and the fourth rule. In one example, presentation of the first score is accompanied by a language-based descriptor or phrase of one or two words.

In different embodiments, a system for providing personalized brain state guidance is disclosed. The system includes a processor and machine-readable media including instructions which, when executed by the processor, cause the processor to perform some or all of the steps described above.

As described herein, the methods and systems allow for automation to create statistically validated feedback for guided meditation sessions and intelligent recommendations for predicting a user's optimal protocol. It can be appreciated the proposed systems and methods can be used to create tailored meditation protocols generally, and/or to provide dynamic, personalized guidance for protocols to replicate any desired brain state. Furthermore, the proposed embodiments provide automated and highly accurate feedback and visualization systems and methods for achieving desired brain state goals. These protocols enable users to more readily maintain a consistent meditation practice in which there is comprehensive analysis and interpretation of their brain activity, guiding each person individually to a deeper practice.

The processes and methods of the embodiments described in this detailed description and shown in the figures can be implemented using any kind of computing system having one or more central processing units (CPUs) and/or graphics processing units (GPUs). The processes and methods of the embodiments could also be implemented using special purpose circuitry such as an application specific integrated circuit (ASIC). The processes and methods of the embodiments may also be implemented on computing systems including read only memory (ROM) and/or random access memory (RAM), which may be connected to one or more processing units. Examples of computing systems and devices include, but are not limited to: servers, cellular phones, smart phones, tablet computers, notebook computers, smart watches, smart glasses, e-book readers, laptop or desktop computers, all-in-one computers, as well as various kinds of digital media players.

The processes and methods of the embodiments can be stored as instructions and/or data on non-transitory computer-readable media. The non-transitory computer readable medium may include any suitable computer readable medium, such as a memory, such as RAM, ROM, flash memory, or any other type of memory known in the art. In some embodiments, the non-transitory computer readable medium may include, for example, an electronic storage device, a magnetic storage device, an optical storage device, an electromagnetic storage device, a semiconductor storage device, or any suitable combination of such devices. More specific examples of the non-transitory computer readable medium may include a portable computer diskette, a floppy disk, a hard disk, magnetic disks or tapes, a read-only memory (ROM), a random access memory (RAM), a static random access memory (SRAM), a portable compact disc read-only memory (CD-ROM), an erasable programmable read-only memory (EPROM or Flash memory), electrically erasable programmable read-only memories (EEPROM), a digital versatile disk (DVD and DVD-ROM), a memory stick, other kinds of solid state drives, and any suitable combination of these exemplary media. A non-transitory computer readable medium, as used herein, is not to be construed as being transitory signals, such as radio waves or other freely propagating electromagnetic waves, electromagnetic waves propagating through a waveguide or other transmission media (e.g., light pulses passing through a fiber-optic cable), or electrical signals transmitted through a wire.

Instructions stored on the non-transitory computer readable medium for carrying out operations of the present invention may be instruction-set-architecture (ISA) instructions, assembler instructions, machine instructions, machine dependent instructions, microcode, firmware instructions, configuration data for integrated circuitry, state-setting data, or source code or object code written in any of one or more programming languages, including an object oriented programming language such as Smalltalk, C++, or suitable language, and procedural programming languages, such as the “C” programming language or similar programming languages.

Aspects of the present disclosure are described in association with figures illustrating flowcharts and/or block diagrams of methods, apparatus (systems), and computing products. It will be understood that each block of the flowcharts and/or block diagrams can be implemented by computer readable instructions. The flowcharts and block diagrams in the figures illustrate the architecture, functionality, and operation of possible implementations of various disclosed embodiments. Accordingly, each block in the flowchart or block diagrams may represent a module, segment, or portion of instructions. In some implementations, the functions set forth in the figures and claims may occur in an alternative order than listed and/or illustrated.

The embodiments may utilize any kind of network for communication between separate computing systems. A network can comprise any combination of local area networks (LANs) and/or wide area networks (WANs), using both wired and wireless communication systems. A network may use various known communications technologies and/or protocols. Communication technologies can include, but are not limited to: Ethernet, 802.11, worldwide interoperability for microwave access (WiMAX), mobile broadband (such as CDMA, and LTE), digital subscriber line (DSL), cable internet access, satellite broadband, wireless ISP, fiber optic internet, as well as other wired and wireless technologies. Networking protocols used on a network may include transmission control protocol/Internet protocol (TCP/IP), multiprotocol label switching (MPLS), User Datagram Protocol (UDP), hypertext transport protocol (HTTP), hypertext transport protocol secure (HTTPS) and file transfer protocol (FTP) as well as other protocols.

Data exchanged over a network may be represented using technologies and/or formats including hypertext markup language (HTML), extensible markup language (XML), Atom, JavaScript Object Notation (JSON), YAML, as well as other data exchange formats. In addition, information transferred over a network can be encrypted using conventional encryption technologies such as secure sockets layer (SSL), transport layer security (TLS), and Internet Protocol security (Ipsec).

Devices for brain data collection such as EEG can employ any available EEG-recording device such as but not limited to headsets with EEG sensors that are typically located inside a headband to be worn on the head. Many vendors produce consumer-grade EEG headbands, including EEG caps, headbands, or headsets produced by NeuroSky Inc.®, Emotiv Inc.®, MacroTellect®, Myndplay®, Neeuro®, FocusCalm®, or Interaxon Inc.® (e.g., providers of the Muse® headset), or other consumer-grade EEG recording devices.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

While various embodiments are described, the description is intended to be exemplary, rather than limiting, and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted.

This disclosure includes and contemplates combinations with features and elements known to the average artisan in the art. The embodiments, features, and elements that have been disclosed may also be combined with any conventional features or elements to form a distinct invention as defined by the claims. Any feature or element of any embodiment may also be combined with features or elements from other inventions to form another distinct invention as defined by the claims. Therefore, it will be understood that any of the features shown and/or discussed in the present disclosure may be implemented singularly or in any suitable combination. Accordingly, the embodiments are not to be restricted except in light of the attached claims and their equivalents. Also, various modifications and changes may be made within the scope of the attached claims.

We claim:

1. A method for generating personalized brain state protocol recommendations, the method comprising:
receiving, at a protocol recommendation system for a meditation application, a dataset representing brainwave activity for a first user in a meditative condition;
accessing, at the protocol recommendation system, a plurality of meditation protocols including at least a first meditation protocol and a second meditation protocol, wherein each meditation protocol promotes a different type of meditation experience;
calculating, by the protocol recommendation system, at least a first depth score characterizing a performance of the dataset with respect to the first meditation protocol and a second depth score characterizing a performance of the dataset with respect to the second meditation protocol, wherein the first depth score is higher than the second depth score; and
presenting, via a user interface for the meditation application and in response to the first depth score being higher than the second depth score, a recommendation to the first user to implement the first meditation protocol.

2. The method of claim 1, further comprising:
receiving, from the first user and via the user interface, a selection of the recommendation; and
providing, via the meditation application, a first guided meditation session that is implemented based on the first meditation protocol.

3. The method of claim 1, further comprising:
presenting, via the meditation application and prior to presenting the recommendation, a first list of options including a first option representing the first meditation protocol and a second option representing the second meditation protocol;
presenting, via the meditation application, a third option for predicting a user's optimal meditation protocol;
receiving, via the user interface and from the first user, a selection of the third option; and
initiating, via the meditation application and in response to the selection of the third option, a first brain activity collection session during which the dataset is obtained.

4. The method of claim 3, further comprising:
presenting, via the user interface and prior to presenting the first list of options, a second list of options including a fourth option representing a first meditation style and a fifth option representing a second meditation style, wherein the first meditation style includes both the first meditation protocol and the second meditation protocol, and the second meditation style includes a third meditation protocol; and
receiving, via the user interface and from the first user, a selection of the fourth option,
wherein presentation of the first list of options is in response to the selection of the fourth option.

5. The method of claim 4, wherein the first meditation style includes meditation protocols for different user experience levels, and the method further comprises:
calculating depth scores for each of the meditation protocols of the first meditation style;
generating a first graph depicting the depth scores, the first graph indicating to which protocol and experience level each depth score corresponds; and
presenting the first graph to a second user who is authorized to access additional data regarding the first user for purposes of coaching.

6. The method of claim 1, wherein the plurality of meditation protocols includes meditation protocols that each promote a different type of meditation experience for the same experience level, and the method further comprises:
calculating depth scores for each of the meditation protocols in the plurality of meditation protocols;
generating a first graph depicting all of the calculated depth scores; and
presenting, via the meditation application, the first graph to the first user.

7. The method of claim 2, wherein the first meditation protocol includes at least a first rule and a second rule, and the method further comprises:
receiving, at the protocol recommendation system, a guided meditation dataset representing brainwave activity for the first user during the first guided meditation session;
calculating, by the protocol recommendation system, a first score characterizing a performance of the guided meditation dataset with respect to the first rule and a second score characterizing a performance of the guided meditation dataset with respect to the second rule;
presenting, via the meditation application, both the first score and the second score;
receiving, via the user interface, a request to improve the first score; and
automatically creating, at the meditation application and in response to the request, a second meditation protocol including only the first rule.

8. The method of claim 2, wherein the first meditation protocol includes at least a first rule and a second rule, and the method further comprises:
receiving, at the protocol recommendation system, a guided meditation dataset representing brainwave activity for the first user during the first guided meditation session;
calculating, by the protocol recommendation system, a first score characterizing a performance of the guided meditation dataset with respect to the first rule and a second score characterizing a performance of the guided meditation dataset with respect to the second rule; and
presenting, via the meditation application, a dashboard including:
a description of the first rule accompanied by the first score, and
a description of the second rule accompanied by the second score.

9. A method of providing personalized brain state guidance, the method comprising:
receiving, at a brainwave guidance system for a meditation application, a first dataset representing brainwave activity for a first user during a first guided meditation session implemented based on a first meditation protocol, the first meditation protocol including at least a first rule and a second rule;
calculating, by the brainwave guidance system, a first score characterizing a performance of the first dataset with respect to the first rule and a second score characterizing a performance of the first dataset with respect to the second rule;
presenting, via the meditation application, both the first score and the second score;
receiving, via a user interface for the meditation application, a first request to improve the first score;
automatically creating, at the meditation application and in response to the first request, a second meditation protocol including only the first rule; and
initiating, at the meditation application, a second guided meditation session for the first user that is implemented based on the second meditation protocol.

10. The method of claim 9, further comprising:
determining, at the meditation application, the first score falls below a predesignated threshold; and
presenting to the first user, in response to the first score falling below the predesignated threshold, a message recommending the creation of the second meditation protocol in order to improve their performance for the first meditation protocol.

11. The method of claim 9, wherein the first rule is directed to brain activity promoting a first metric and the second rule is directed to brain activity promoting a different, second metric.

12. The method of claim 11, wherein the first metric represents one of power, percent of total power, power ratio, coherence, connectivity, minimum frequency, maximum frequency, phase synchrony, complexity, brain location, and target brainwave direction.

13. The method of claim 9, further comprising:
receiving, at the brainwave guidance system, a second dataset representing brainwave activity for the first user during a second guided meditation session implemented based on a third meditation protocol, the third meditation protocol including at least a third rule, fourth rule, and a fifth rule;
calculating, by the brainwave guidance system, a third score characterizing a performance of the second dataset with respect to the third rule, a fourth score characterizing a performance of the second dataset with respect to the fourth rule, and a fifth score characterizing a performance of the second dataset with respect to the fifth rule;
presenting, via the meditation application, the third score, fourth score, and the fifth score;
receiving, via the user interface, a second request to improve both the fourth score and the fifth score; and
automatically creating, at the meditation application and in response to the second request, a fourth meditation protocol including only the fourth rule and the fifth rule.

14. The method of claim 9, further comprising:
receiving, at the brainwave guidance system, a second dataset representing brainwave activity for the first user during a second guided meditation session implemented based on a third meditation protocol, the third meditation protocol including at least a third rule, fourth rule, and a fifth rule;
calculating, by the brainwave guidance system, a third score characterizing a performance of the second dataset with respect to the third rule, a fourth score characterizing a performance of the second dataset with respect to the fourth rule, and a fifth score characterizing a performance of the second dataset with respect to the fifth rule;
determining, at the meditation application, both the third score and the fourth score fall below a predesignated threshold, while the fifth score is above the predesignated threshold; and
automatically creating, at the meditation application and in response to the third score and the fourth score falling below the predesignated threshold, a fourth meditation protocol including only the third rule and the fourth rule.

15. The method of claim 9, wherein presentation of the first score is accompanied by a language-based descriptor.

16. A method of providing personalized meditation feedback, the method comprising:
receiving, at a brainwave feedback system for a meditation application, a first meditation dataset representing brainwave activity for a first user during a first guided meditation session implemented based on a first meditation protocol, the first meditation protocol including at least a first rule and a second rule;
calculating, by the brainwave feedback system, a first score characterizing a performance of the first meditation dataset with respect to the first rule and a second score characterizing a performance of the first meditation dataset with respect to the second rule;
presenting, via the meditation application, a dashboard including:
a description of the first rule accompanied by the first score, and
a description of the second rule accompanied by the second score.

17. The method of claim 16, wherein the dashboard further includes a first text-based descriptor accompanying the first score and a second text-based descriptor accompanying the second score.

18. The method of claim 16, wherein the first rule is directed to brain activity promoting a first metric and the second rule is directed to brain activity promoting a different, second metric.

19. The method of claim 18, further comprising:
accessing, at the meditation application, a frequency band power database that includes a first set of text-based descriptions interpreting a range of scores possible for the first metric, and a second set of text-based descriptions interpreting a range of scores possible for the second metric;
selecting a first text-based description linked to the first score from the first set and selecting a second text-based description linked to the second score from the second set; and
presenting, via the meditation application, both the first text-based description and the second text-based description.

20. The method of claim 16, further comprising:
receiving, via a user interface for the meditation application, a first request to improve the first score;
automatically creating, at the meditation application and in response to the first request, a second meditation protocol including only the first rule; and
initiating, at the meditation application, a second guided meditation session for the first user that is implemented based on the second meditation protocol.
